SEQUENCE LISTING (33 sequences)

SEQ ID NO: 1 - protein, 244 aa, Artificial
  ULBP3_AA
MAAAASPAILPRLAILPYLLFDWSGTGRADAHSLWYNFTIIHLPRHGQQWCEVQSQVDQKNFLSYDCGSDKVLSMGHLEEQLYATDAWGKQLEMLREVGQRLRLELADTELEDFTPSGPLTLQVRMSCECEADGYIRGSWQFSFDGRKFLLFDSNNRKWTVVHAGARRMKEKWEKDSGLTTFFKMVSMRDCKSWLRDFLMHRKKRLEPTAPPTMAPGLAQPKAIATTLSPWSFLIILCFILPGI

SEQ ID NO: 2 - DNA, 735 bp, Artificial
  ULBP3_NT
atggcagcggccgccagccccgcgatccttccgcgcctcgcgattcttccgtacctgctattcgactggtccgggacggggcgggccgacgctcactctctctggtataacttcaccatcattcatttgcccagacatgggcaacagtggtgtgaggtccagagccaggtggatcagaagaattttctctcctatgactgtggcagtgacaaggtcttatctatgggtcacctagaagagcagctgtatgccacagatgcctggggaaaacaactggaaatgctgagagaggtggggcagaggctcagactggaactggctgacactgagctggaggatttcacacccagtggacccctcacgctgcaggtcaggatgtcttgtgagtgtgaagccgatggatacatccgtggatcttggcagttcagcttcgatggacggaagttcctcctctttgactcaaacaacagaaagtggacagtggttcacgctggagccaggcggatgaaagagaagtgggagaaggatagcggactgaccaccttcttcaagatggtctcaatgagagactgcaagagctggcttagggacttcctgatgcacaggaagaagaggctggaacccacagcaccacccaccatggccccaggcttagctcaacccaaagccatagccaccaccctcagtccctggagcttcctcatcatcctctgcttcatcctccctggcatctga

SEQ ID NO: 3 - protein, 246 aa, Artificial
  ULBP6_AA
MAAAAIPALLLCLPLLFLLFGWSRARRDDPHSLCYDITVIPKFRPGPRWCAVQGQVDEKTFLHYDCGNKTVTPVSPLGKKLNVTMAWKAQNPVLREVVDILTEQLLDIQLENYTPKEPLTLQARMSCEQKAEGHSSGSWQFSIDGQTFLLFDSEKRMWTTVHPGARKMKEKWENDKDVAMSFHYISMGDCIGWLEDFLMGMDSTLEPSAGAPLAMSSGTTQLRATATTLILCCLLIILPCFILPGI

SEQ ID NO: 4 - DNA, 741 bp, Artificial
  ULBP6_NT
atggcagcagccgccatcccagctttgcttctgtgcctcccgcttctgttcctgctgttcggctggtcccgggctaggcgagacgaccctcactctctttgctatgacatcaccgtcatccctaagttcagacctggaccacggtggtgtgcggttcaaggccaggtggatgaaaagacttttcttcactatgactgtggcaacaagacagtcacacccgtcagtcccctggggaagaaactaaatgtcacaatggcctggaaagcacagaacccagtactgagagaggtggtggacatacttacagagcaactgcttgacattcagctggagaattacacacccaaggaacccctcaccctgcaggcaaggatgtcttgtgagcagaaagctgaaggacacagcagtggatcttggcagttcagtatcgatggacagaccttcctactctttgactcagagaagagaatgtggacaacggttcatcctggagccagaaagatgaaagaaaagtgggagaatgacaaggatgtggccatgtccttccattacatctcaatgggagactgcataggatggcttgaggacttcttgatgggcatggacagcaccctggagccaagtgcaggagcaccactcgccatgtcctcaggcacaacccaactcagggccacagccaccaccctcatcctttgctgcctcctcatcatcctcccctgcttcatcctccctggcatctga

SEQ ID NO: 5 - protein, 383 aa, Artificial
  MICA_AA
MGLGPVFLLLAGIFPFAPPGAAAEPHSLRYNLTVLSWDGSVQSGFLTEVHLDGQPFLRCDRQKCRAKPQGQWAEDVLGNKTWDRETRDLTGNGKDLRMTLAHIKDQKEGLHSLQEIRVCEIHEDNSTRSSQHFYYDGELFLSQNLETKEWTMPQSSRAQTLAMNVRNFLKEDAMKTKTHYHAMHADCLQELRRYLKSGVVLRRTVPPMVNVTRSEASEGNITVTCRASGFYPWNITLSWRQDGVSLSHDTQQWGDVLPDGNGTYQTWVATRICQGEEQRFTCYMEHSGNHSTHPVPSGKVLVLQSHWQTFHVSAVAAAAIFVIIIFYVRCCKKKTSAAEGPELVSLQVLDQHPVGTSDHRDATQLGFQPLMSDLGSTGSTEGA

SEQ ID NO: 6 - DNA, 1152 bp, Artificial
  MICA_NT
atggggctgggcccggtcttcctgcttctggctggcatcttcccttttgcacctccgggagctgctgctgagccccacagtcttcgttataacctcacggtgctgtcctgggatggatctgtgcagtcagggtttctcactgaggtacatctggatggtcagcccttcctgcgctgtgacaggcagaaatgcagggcaaagccccagggacagtgggcagaagatgtcctgggaaataagacatgggacagagagaccagagacttgacagggaacggaaaggacctcaggatgaccctggctcatatcaaggaccagaaagaaggcttgcattccctccaggagattagggtctgtgagatccatgaagacaacagcaccaggagctcccagcatttctactacgatggggagctcttcctctcccaaaacctggagactgaggaatggacaatgccccagtcctccagagctcagaccttggccatgaacgtcaggaatttcttgaaggaagatgccatgaagaccaagacacactatcacgctatgcatgcagactgcctgcaggaactacggcgatatctaaaatccggcgtagtcctgaggagaacagtgccccccatggtgaatgtcacccgcagcgaggcctcagagggcaacattaccgtgacatgcagggcttctggcttctatccctggaatatcacactgagctggcgtcaggatggggtatctttgagccacgacacccagcagtggggggatgtcctgcctgatgggaatggaacctaccagacctgggtggccaccaggatttgccaaggagaggagcagaggttcacctgctacatggaacacagcgggaatcacagcactcaccctgtgccctctgggaaagtgctggtgcttcagagtcattggcagacattccatgtttctgctgttgctgctgctgctatttttgttattattattttctatgtccgttgttgtaagaagaaaacatcagctgcagagggtccagagctcgtgagcctgcaggtcctggatcaacacccagttgggacgagtgaccacagggatgccacacagctcggatttcagcctctgatgtcagatcttgggtccactggctccactgagggcacctag

SEQ ID NO: 7 - protein, 253 aa, Artificial
  RAE1_AA
MAKAAVTKRHHFMIQKLLILLSYGYTNGLDDAHSLRCNLTIKDPTPADPLWYEAKCFVGEILILHLSNINKTMTSGDPGETANATEVKKCLTQPLKNLCQKLRNKVSNTKVDTHKTNGYPHLQVTMIYPQSQGRTPSATWEFNISDSYFFTFYTENMSWRSANDESGVIMNKWKDDGEFVKQLKFLIHECSQKMDEFLKQSKEKPRSTSRSPSITQLTSTSPLPPPSHSTSKKGFISVGLIFISLLFAFAFAM

SEQ ID NO: 8 - DNA, 762 bp, Artificial
  RAE1_NT
atggccaaggcagcagtgaccaagcgccatcattttatgattcagaagctgttaattctactgagctatggatacaccaacgggctggatgatgcacactctcttaggtgcaacttgaccatcaaggatcctaccccagcagatcctctctggtatgaagcgaagtgcttcgtgggtgaaatacttatcctccatttaagtaacataaacaagaccatgacttcaggtgacccaggggagacagcaaatgccactgaagtgaagaaatgtttgacacaacctctgaaaaatttgtgccagaagttgaggaacaaggtgtctaacaccaaagtggacactcacaagaccaatggttacccacatttacaagtcaccatgatttatccgcaaagccagggccgaactcctagtgccacctgggaattcaacatcagtgacagttacttcttcaccttctacacagagaatatgagctggagatcagctaatgatgaatcaggggttatcatgaataaatggaaagatgatggggaatttgtgaaacaattgaaattcttgatacacgaatgcagtcagaaaatggatgaattcttaaagcagtccaaggaaaagccaagatcaacctcaaggtcccccagtatcacccagcttacatcaacttccccgcttccacctcccagccactctacttctaagaaaggatttatctctgtgggactcatcttcatatctttattatttgcatttgcatttgcgatgtga

SEQ ID NO: 9 - protein, 262 aa, Artificial
  H60A_AA
MAKGATSKSNHCLILSLFILLSYLGTILADGTDSLSCELTFNYRNLHGQCSVNGKTLLDFGDKKHEENATKMCADLSQNLREISEEMWKLQSGNDTLNVTTQSQYNQGKFIDGFWAINTDEQHSIYFYPLNMTWRESHSDNSSAMEQWKNKNLEKDMRNFLITYFSHCLNKSSSHFREMPKSTLKVPDTTQRTNATQIHPTVNNFRHNSDNQGLSVTWIVIICIGGLVSFMAFMVFAWCMLKKKKKRCSLLLLFFCSPHYQL

SEQ ID NO: 10 - DNA, 789 bp, Artificial
  H60A_NT
atggcaaagggagccaccagcaagagcaaccattgcctgattctgagccttttcattctgctgagctatctggggaccatactggcagatggtacagactctctaagttgtgaattaactttcaactatcgtaatctacatggacagtgctcagtgaatggaaagactctccttgattttggtgataaaaaacatgaggaaaatgctactaagatgtgtgctgatttgtcccaaaacctgagagagatttcagaagagatgtggaagttacaatcaggtaatgataccttgaatgtcacaacacaatctcagtataatcaaggaaaattcattgatggattctgggccatcaacactgatgaacagcatagcatctacttttatccacttaatatgacctggagagaaagtcattctgataacagcagtgccatggagcagtggaagaacaagaacctagagaaagatatgaggaatttcctcatcacatatttcagtcactgcctcaacaaatcgtcatcacactttagagaaatgccaaaatcaacattaaaggtgccggataccacccaacgtacaaatgccactcagattcatcctacagtgaataacttccgacataattctgacaaccagggtctgagtgtcacctggattgtgattatatgtataggaggattagtgtctttcatggcattcatggtattcgcttggtgtatgctgaagaaaaaaaaaaaaaggtgctccctgctgctcctcttcttctgcagtcctcattaccagctatga

SEQ ID NO: 11 - DNA, 4107 bp, Artificial
  Cas9-coding sequence
atggacaagaagtacagcatcggcctggacatcggtaccaacagcgtgggctgggccgtgatcaccgacgagtacaaggtgcccagcaagaagttcaaggtgctgggcaacaccgaccgccacagcatcaagaagaacctgatcggcgccctgctgttcgacagcggcgagaccgccgaggccacccgcctgaagcgcaccgcccgccgccgctacacccgccgcaagaaccgcatctgctacctgcaggagatcttcagcaacgagatggccaaggtggacgacagcttcttccaccgcctggaggagagcttcctggtggaggaggacaagaagcacgagcgccaccccatcttcggcaacatcgtggacgaggtggcctaccacgagaagtaccccaccatctaccacctgcgcaagaagctggtggacagcaccgacaaggccgacctgcgcctgatctacctggccctggcccacatgatcaagttccgcggccacttcctgatcgagggcgacctgaaccccgacaacagcgacgtggacaagctgttcatccagctggtgcagacctacaaccagctgttcgaggagaaccccatcaacgccagcggcgtggacgccaaggccatcctgagcgcccgcctgagcaagagccgccgcctggagaacctgatcgcccagctgcccggcgagaagaagaacggcctgttcggcaacctgatcgccctgagcctgggcctgacccccaacttcaagagcaacttcgacctggccgaggacgccaagctgcagctgagcaaggacacctacgacgacgacctggacaacctgctggcccagatcggcgaccagtacgccgacctgttcctggccgccaagaacctgagcgacgccatcctgctgagcgacatcctgcgcgtgaacaccgagatcaccaaggcccccctgagcgccagcatgatcaagcgctacgacgagcaccaccaggacctgaccctgctgaaggccctggtgcgccagcagctgcccgagaagtacaaggagatcttcttcgaccagagcaagaacggctacgccggctacatcgacggcggcgccagccaggaggagttctacaagttcatcaagcccatcctggagaagatggacggcaccgaggagctgctggtgaagctgaaccgcgaggacctgctgcgcaagcagcgcaccttcgacaacggcagcatcccccaccagatccacctgggcgagctgcacgccatcctgcgccgccaggaggacttctaccccttcctgaaggacaaccgcgagaagatcgagaagatcctgaccttccgcatcccctactacgtgggccccctggcccgcggcaacagccgcttcgcctggatgacccgcaagagcgaggagaccatcaccccctggaacttcgaggaggtggtggacaagggcgccagcgcccagagcttcatcgagcgcatgaccaacttcgacaagaacctgcccaacgagaaggtgctgcccaagcacagcctgctgtacgagtacttcaccgtgtacaacgagctgaccaaggtgaagtacgtgaccgagggcatgcgcaagcccgccttcctgagcggcgagcagaagaaggccatcgtggacctgctgttcaagaccaaccgcaaggtgaccgtgaagcagctgaaggaggactacttcaagaagatcgagtgcttcgacagcgtggagatcagcggcgtggaggaccgcttcaacgccagcctgggcacctaccacgacctgctgaagatcatcaaggacaaggacttcctggacaacgaggagaacgaggacatcctggaggacatcgtgctgaccctgaccctgttcgaggaccgcgagatgatcgaggagcgcctgaagacctacgcccacctgttcgacgacaaggtgatgaagcagctgaagcgccgccgctacaccggctggggccgcctgagccgcaagcttatcaacggcatccgcgacaagcagagcggcaagaccatcctggacttcctgaagagcgacggcttcgccaaccgcaacttcatgcagctgatccacgacgacagcctgaccttcaaggaggacatccagaaggcccaggtgagcggccagggcgacagcctgcacgagcacatcgccaacctggccggcagccccgccatcaagaagggcatcctgcagaccgtgaaggtggtggacgagctggtgaaggtgatgggccgccacaagcccgagaacatcgtgatcgagatggcccgcgagaaccagaccacccagaagggccagaagaacagccgcgagcgcatgaagcgcatcgaggagggcatcaaggagctgggcagccagatcctgaaggagcaccccgtggagaacacccagctgcagaacgagaagctgtacctgtactacctgcagaacggccgcgacatgtacgtggaccaggagctggacatcaaccgcctgagcgactacgacgtggaccacatcgtgccccagagcttcctgaaggacgacagcatcgacaacaaggtgctgacccgcagcgacaagaaccgcggcaagagcgacaacgtgcccagcgaggaggtggtgaagaagatgaagaactactggcgccagctgctgaacgccaagctgatcacccagcgcaagttcgacaacctgaccaaggccgagcgcggcggcctgagcgagctggacaaggccggcttcatcaagcgccagctggtggagacccgccagatcaccaagcacgtggcccagatcctggacagccgcatgaacaccaagtacgacgagaacgacaagctgatccgcgaggtgaaggtgatcaccctgaagagcaagctggtgagcgacttccgcaaggacttccagttctacaaggtgcgcgagatcaacaactaccaccacgcccacgacgcctacctgaacgccgtggtgggcaccgccctgatcaagaagtaccccaagctggagagcgagttcgtgtacggcgactacaaggtgtacgacgtgcgcaagatgatcgccaagagcgagcaggagatcggcaaggccaccgccaagtacttcttctacagcaacatcatgaacttcttcaagaccgagatcaccctggccaacggcgagatccgcaagcgccccctgatcgagaccaacggcgagaccggcgagatcgtgtgggacaagggccgcgacttcgccaccgtgcgcaaggtgctgagcatgccccaggtgaacatcgtgaagaagaccgaggtgcagaccggcggcttcagcaaggagagcatcctgcccaagcgcaacagcgacaagctgatcgcccgcaagaaggactgggaccccaagaagtacggcggcttcgacagccccaccgtggcctacagcgtgctggtggtggccaaggtggagaagggcaagagcaagaagctgaagagcgtgaaggagctgctgggcatcaccatcatggagcgcagcagcttcgagaagaaccccatcgacttcctggaggccaagggctacaaggaggtgaagaaggacctgatcatcaagctgcccaagtacagcctgttcgagctggagaacggccgcaagcgcatgctggccagcgccggcgagctgcagaagggcaacgagctggccctgcccagcaagtacgtgaacttcctgtacctggccagccactacgagaagctgaagggcagccccgaggacaacgagcagaagcagctgttcgtggagcagcacaagcactacctggacgagatcatcgagcagatcagcgagttcagcaagcgcgtgatcctggccgacgccaacctggacaaggtgctgagcgcctacaacaagcaccgcgacaagcccatccgcgagcaggccgagaacatcatccacctgttcaccctgaccaacctgggcgcccccgccgccttcaagtacttcgacaccaccatcgaccgcaagcgctacaccagcaccaaggaggtgctggacgccaccctgatccaccagagcatcaccggtctgtacgagacccgcatcgacctgagccagctgggcggcgactaa

SEQ ID NO: 12 - protein, 1368 aa, Artificial [sequence omitted]

SEQ ID NO: 13 - protein, 23 aa, Artificial
  HA2
GLFGAIAGFIENGWEGMIDGWYG

SEQ ID NO: 14 - protein, 18 aa, Artificial
  CM18
KWKLFKKIGAVLKVLTTG

SEQ ID NO: 15 - protein, 15 aa, Artificial
  S10
KWKLARAFARAIKKL

SEQ ID NO: 16 - DNA, 20 bp, Artificial
  hTGFBR2_crRNA#7
ggccgctgcacatcgtcctg

SEQ ID NO: 17 - DNA, 20 bp, Artificial
  hTGFBR2_crRNA#8
cccaccgcacgttcagaagt

SEQ ID NO: 18 - DNA, 20 bp, Artificial
  hTGFBR2_crRNA#9
ccgacttctgaacgtgcggt

SEQ ID NO: 19 - DNA, 20 bp, Artificial
  hTGFBR2_crRNA#2
cccctaccatgactttattc

SEQ ID NO: 20 - DNA, 20 bp, Artificial
  hTGFBR2_crRNA#10
ccgcgtcttgccggtttccc

SEQ ID NO: 21 - DNA, 20 bp, Artificial
  hTGFBR2_crRNA#ref
cctgagcagcccccgaccca

SEQ ID NO: 22 - DNA, 33 bp, Artificial
  ULBP3_F
cccggtctcctaaggacgctcactctctctggt

SEQ ID NO: 23 - DNA, 34 bp, Artificial
  ULBP3_R
cccggtctccccatttccagcctcttcttcctgt

SEQ ID NO: 24 - DNA, 34 bp, Artificial
  ULBP6_F
cccggtctcctaaggaccctcactctctttgcta

SEQ ID NO: 25 - DNA, 33 bp, Artificial
  ULBP6_R
cccggtctccccatgctgtccatgcccatcaag

SEQ ID NO: 26 - DNA, 31 bp, Artificial
  MICA_F
cccggtctcctaagatggggctgggcccggt

SEQ ID NO: 27 - DNA, 33 bp, Artificial
  MICA_R
cccggtctccccatagagggcacagggtgagtg

SEQ ID NO: 28 - DNA, 34 bp, Artificial
  RAE1_F
cccggtctcctaaggacgcccatagtttacgctg

SEQ ID NO: 29 - DNA, 37 bp, Artificial
  RAE1_R
cccggtctccccattttctctttcgattgtttcagaa

SEQ ID NO: 30 - DNA, 34 bp, Artificial
  H60a_F
cccggtctcctaagcttttgtcgtaccttggcac

SEQ ID NO: 31 - DNA, 35 bp, Artificial
  H60a_R
cccggtctccccatgaggccttgattatcactgtt

SEQ ID NO: 32 - DNA, 31 bp, Artificial
  Cas9-NLS_pET21a_F
cccggtctccatggataagcatcaccaccac

SEQ ID NO: 33 - DNA, 31 bp, Artificial
  Cas9-NLS_pET21a_R
cccggtctcccttatccatcaccttcctctt

(19) United States
(12) Patent Application Publication
(10) Pub. No.: US 2022/0243186 A1
(43) Pub. Date: Aug. 4, 2022

(54) FUSION PROTEIN FOR NATURAL KILLER CELL SPECIFIC CRISPR/CAS SYSTEM AND USE THEREOF

(71) Applicant: KOREA INSTITUTE OF SCIENCE AND TECHNOLOGY, Seoul (KR)

(72) Inventors: Mihue JANG, Seoul (KR); Junghee KIM, Seoul (KR); Sang Chul SHIN, Seoul (KR); Young Eun LEE, Seoul (KR); Han-Na YOON, Seoul (KR)

(73) Assignee: KOREA INSTITUTE OF SCIENCE AND TECHNOLOGY, Seoul (KR)

(21) Appl. No.: 17/544,429

(22) Filed: Dec. 7, 2021

(30) Foreign Application Priority Data
Feb. 3, 2021 (KR) 10-2021-0015238

(51) Int. Cl. C12N 9/22; C12N 15/10; C12N 15/113; C12N 15/63

(52) U.S. Cl. CPC C12N 9/22; C12N 15/102; C12N 2310/20; C12N 15/63; C12N 15/113

(57) ABSTRACT

Provided are a fusion protein used in a CRISPR/Cas system, and a complex including the same and use thereof. Since the gene editing complex including the fusion protein of the present disclosure includes an NKG2D ligand capable of binding to NKG2D expressed on the membrane of natural killer cells, it may be specifically delivered to NKG2D receptor-expressing cells or natural killer cells. Since it may be effectively delivered into the cells through endocytosis of NKG2D without a carrier, a target gene or a target DNA of NKG2D receptor-expressing cells or natural killer cells may be manipulated using the complex.

### CROSS-REFERENCE TO RELATED APPLICATION

This application is based on and claims priority under 35 U.S.C. § 119 to Korean Patent Application No. 10-2021-0015238, filed on Feb. 3, 2021, in the Korean Intellectual Property Office, the disclosure of which is incorporated by reference herein in its entirety.

### BACKGROUND

### 1. Field

The present disclosure relates to a fusion protein used in a CRISPR/Cas system, a complex including the same, and use thereof.

### 2. Description of the Related Art

RNA-programmed Cas9 ribonucleoprotein (Cas9 RNPs) system is a complex including Cas9 protein and a chimeric single guide RNA (sgRNA) and is able to perform genome editing. sgRNA consists of CRISPR RNA (crRNA) and transactivating crRNA (tracrRNA). Specifically, crRNA plays a role in targeting a desired genomic sequence to form base-pairing, and tracrRNA complementarily binds to some base sequences of crRNA to allow Cas9 to cleave DNA. Cas9 RNP system delivers RNA or a protein directly to cells without additional steps, for example, transcription and translation, and therefore, use of CRISPR-Cas9 system with purified Cas9 RNPs provides an innovative platform for highly efficient genome editing with relatively few off-target cleavages, as compared with plasmid or plasmid or virus-mediated delivery of sgRNAs. In general, Cas9 RNP-mediated delivery to target cells is performed via lipid-mediated transfection, nanoparticles, or electroporation.

It has been reported that cationic lipid-mediated delivery of Cas9 RNP and sgRNA achieved up to 20% genome modification in the mouse inner ear in vivo, when complexed with 50% RNAiMAX or Lipofetamine 2000.1. Recently, an engineered Cas9 with multiple SV40 nuclear localization sequences for gene editing in the mouse brain in vivo has been reported. Nevertheless, Cas9 RNP-mediated in vivo gene editing remains challenging. In particular, since Cas9 RNP has no intracellular transduction activity, direct complex formation in vivo and cell internalization are achieved through conjugation with cationic polymers or lipid carriers, and some limitations remain with regard to release of payloads into cytoplasm, nuclear localization, and safety.

On the other hand, natural killer (NK) cells are cytotoxic lymphocytes that constitute a major component of an innate immune system. NK cells, which generally represent about 10% to about 15% of circulating lymphocytes, bind to and kill targeted cells including virus-infected cells and many malignant cells, non-specifically to antigens and without prior immune sensitization.

With regard to cancer, phenotypic changes that distinguish tumor cells from normal cells derived from the same tissue are often associated with one or more changes in expression of a particular gene product, which include loss of normal cell surface components or acquisition of others (i.e., antigens undetectable in corresponding normal, non-cancerous tissues). Antigens expressed in neoplastic or tumor cells, but not in normal cells, or expressed in neoplastic cells at levels substantially exceeding those found in normal cells are called “tumor-specific antigens” or “tumor-associated antigens”. Such tumor-specific antigens may serve as markers for tumor phenotypes.

These tumor-specific antigens have been used as targets for cancer immunotherapy. This therapy improves cytotoxicity to cancer cells by using a chimeric antigen receptor (CAR) expressed on the surface of immune cells, including T cells and NK cells, and thus there is a need for an effective gene manipulation technique for NK cells.

Accordingly, the present inventors have developed a fusion protein that may form a complex with guide RNA in the CRISPR/Cas system to deliver it into cells without a cationic polymer or lipid carrier, and they found that the complex including the fusion protein is delivered to natural killer cells to perform gene editing, thereby completing the present disclosure.

### SUMMARY

One aspect provides a fusion protein including a CRISPR-associated protein (Cas protein) and an NKG2D ligand-derived protein.

Another aspect provides a polynucleotide encoding the fusion protein.

Still another aspect provides an expression vector including the polynucleotide.

Still another aspect provides a gene editing complex including the fusion protein including the Cas protein and the NKG2D ligand-derived protein; and guide RNA.

Still another aspect provides a composition for editing a target DNA or a target gene specifically to NKG2D receptor-expressing cells or natural killer cells, the composition including the gene editing complex.

Additional aspects will be set forth in part in the description which follows and, in part, will be apparent from the description, or may be learned by practice of the presented embodiments of the disclosure.

An aspect provides a fusion protein including a CRISPR-associated protein (Cas protein) and an NKG2D ligand-derived protein.

As used herein, the term “NKG2D” is a transmembrane protein belonging to the C-type lectin-like receptors of NKG2 family, and is encoded by KLRK1 gene which is located in the NK-gene complex (NKC). In mice, NKG2D is known to be expressed in NK cells, NK1.1+T cells, activated CD8+αβT cells, activated macrophages, etc. In humans, NKG2D is known to be expressed in NK cells, CD8+αβT cells, etc.

The NKG2D ligand means a protein binding to NKG2D, and it is known that the NKG2D ligand is present at only low levels on surface of normal cells, but overexpressed by cancer, infection, transformation, aging, stress, etc. The NKG2D ligand may be specifically one or more selected from the group consisting of UL16 binding protein 3 (ULBP3), MHC Class I Polypeptide-Related Sequence A (MICA), ULBP6, Retinoic acid early-inducible protein 1-beta (RAE1), histocompatibility antigen 60a (H60A), MICB, ULBP1, ULBP2, ULBP4, ULBP5, RAE1α, RAE1γ, RAE1δ, RAE1ϵ, and H60B.

The ULBP3 (Expasy No: Q9BZM4) consists of an amino acid sequence of SEQ ID NO: 1, and is encoded by a nucleotide sequence of SEQ ID NO: 2. Further, the ULBP3-derived protein included in the fusion protein may include an amino acid sequence at positions 30 to 207 of SEQ ID NO: 1.

The ULBP6 (Expasy No: Q5VY80) consists of an amino acid sequence of SEQ ID NO: 3, and is encoded by a nucleotide sequence of SEQ ID NO: 4. Further, the ULBP6-derived protein included in the fusion protein may include an amino acid sequence at positions 29 to 203 of SEQ ID NO: 3.

The MICA (Expasy No: Q29983) consists of an amino acid sequence of SEQ ID NO: 5, and is encoded by a nucleotide sequence of SEQ ID NO: 6. Further, the MICA-derived protein included in the fusion protein may include an amino acid sequence at positions 1 to 297 of SEQ ID NO: 5.

The RAE1 (Expasy No: O08603) consists of an amino acid sequence of SEQ ID NO: 7, and is encoded by a nucleotide sequence of SEQ ID NO: 8. Further, the RAE1-derived protein included in the fusion protein may include an amino acid sequence at positions 31 to 204 of SEQ ID NO: 7.

The H60A (Expasy No: Q3TDZ7) consists of an amino acid sequence of SEQ ID NO: 9, and is encoded by a nucleotide sequence of SEQ ID NO: 10. Further, the H60A-derived protein included in the fusion protein may include an amino acid sequence at positions 20 to 214 of SEQ ID NO: 9.

In general, “CRISPR system” collectively refers to transcripts and other elements involved in the expression of or directing the activity of CRISPR-associated (“Cas”) genes, including sequences encoding a Cas gene, a trans-activating CRISPR) sequence (tracr (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), guide RNA, or other sequences and transcripts from a CRISPR locus. In some embodiments, one or more elements of the CRISPR system are derived from a type I, type II, or type III CRISPR system. In some embodiments, one or more elements of the CRISPR system are derived from a particular organism including an endogenous CRISPR system, e.g., Streptococcus pyogenes. In general, the CRISPR system is characterized by elements that promote formation of a CRISPR complex at the site of a target sequence (also referred to as a protospacer in the context of an endogenous CRISPR system).

The NKG2D ligand may be introduced into cells via endocytosis by a receptor-ligand interaction with NKG2D, and thus the fusion protein including the NKG2D ligand or a complex including the fusion protein may be effectively introduced into cells expressing NKG2D on the surface thereof without a carrier.

In the context of formation of the CRISPR complex, the “target DNA” or “target gene” refers to a sequence to which a guide sequence is designed to have complementarity, where hybridization between a target sequence and a guide sequence promotes formation of the CRISPR complex. Full complementarity is not necessarily required, but there is sufficient complementarity to cause hybridization and to promote formation of the CRISPR complex. The target sequence may include any polynucleotide, e.g., DNA or RNA polynucleotides. In some embodiments, the target sequence is located in the nucleus or cytoplasm of cells. In some embodiments, the target sequence may be located in organelles of eukaryotic cells, e.g., mitochondria or chloroplast.

When the Cas protein forms a complex with two RNAs called CRISPR RNA (crRNA) and trans-activating crRNA (tracrRNA), it forms active endonuclease or nickase. Non-limiting examples of the Cas protein include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, or modified versions thereof. These enzymes are known; for example, an amino acid sequence of Streptococcus pyogenes Cas9 protein may be obtained from the SwissProt database under accession number Q99ZW2. In some embodiments, the unmodified CRISPR enzyme, e.g., Cas9 has DNA cleavage activity. In some embodiments the CRISPR enzyme is Cas9, and may be Cas9 from Streptococcus pyogenes or Streptococcus pneumoniae. In some embodiments, the Cas protein is codon-optimized for expression in eukaryotic cells. The coding sequence of Cas9 may include, for example, a polynucleotide sequence having 80% or more homology to a polynucleotide sequence of SEQ ID NO: 11. Further, Cas9 may include an amino acid sequence of SEQ ID NO: 12 derived from Streptococcus pyogenes.

The RNA-guided CRISPR associated nuclease Cas9 provides an epoch-making technology for target gene knock-out, transcriptional activation, and inhibition using single guide RNA (sgRNA) (i.e., crRNA-tracrRNA fusion transcript), and this technology is known to target numerous gene locations.

The Cas protein may be Cas9, Cpf1, or a Cas variant protein, and the Cas9 (or Cpf1) protein refers to an essential protein element in the CRISPR/Cas9 system, and information on the Cas9 (or Cpf1) gene and protein may be obtained from GenBank of the national center for biotechnology information (NCBI), but is not limited thereto. With regard to the CRISPR-associated gene encoding Cas (or Cpf1) protein, about 40 or more different Cas (or Cpf1) protein families are known to exist, and according to a specific combination of cas gene and repeat structure, 8 CRISPR subtypes (Ecoli, Ypest, Nmeni, Dvulg, Tneap, Hmari, Apern, and Mtube) may be defined. Therefore, each of the CRISPR subtypes may form a repeating unit to form a polyribonucleotide-protein complex.

Gene manipulation artificially performed to reduce expression or activity of DNA or a gene may be induced by Cas9 protein, Cpf1 protein, or Cas variant protein. The Cas9 protein or Cpf1 protein applicable to the gene manipulation may be one or more selected from the group consisting of Streptococcus pyogenes-derived Cas9 protein, Campylobacter jejuni-derived Cas9 protein, Streptococcus thermophilus-derived Cas9 protein, Streptococcus aureus-derived Cas9 protein, Neisseria meningitidis-derived Cas9 protein, and Cpf1 protein. When the Cas9 (or Cpf1) is encoded by DNA and delivered to an individual or cell, the DNA may generally include a regulatory element (e.g., a promoter) operable in target cells (but not necessarily). A promoter for Cas9 (or Cpf1) expression may be, for example, a CMV, EF-I a, EFS, MSCV, PGK, or CAG promoter. A promoter for gRNA expression may be, for example, an HI, EF-Ia, tRNA or U6 promoter. The Cas9 (or Cpf1)-coding sequence may include a nuclear localization signal (NLS) (e.g., SV40 NLS). In one embodiment, the promoter may have tissue specificity or cell specificity.

The fusion protein may further include a nuclear localization sequence (NLS).

As used herein, the term “nuclear localization sequence or signal (NLS)” refers to an amino acid sequence that serves to transport a specific substance (e.g., protein) into the cell nucleus, generally, through nuclear pores (Kalderon D, et al., Cell 39:499509 (1984); Dingwall C, et al., J Cell Biol. 107(3):8419(1988)). The nuclear localization sequence is not necessary for activity of the CRISPR complex in eukaryotes, but by including such sequences, activity of the system is enhanced, especially, to target nucleic acid molecules in the nucleus. The CRISPR enzyme may include one or more nuclear localization sequences of sufficient strength to drive accumulation of the CRISPR enzyme in a detectable amount in the nucleus of a eukaryotic cell.

The nuclear localization sequence may be linked to the C-terminus of the Cas9 protein. Specifically, the nuclear localization sequence may be linked to the C-terminus of the Cas9 protein and may be linked to the N-terminus of the NKG2D ligand-derived protein.

The fusion protein may further include an endosomal escape peptide (EEP).

As used herein, the term “endosomal escape” refers to escape of a substance carried in an endosome from the endosome by a method such as increasing the osmotic pressure inside the endosome, destabilizing the endosome membrane, etc. In general, physiologically active substances outside cells are internalized into cells through formation of cell organelles called endosomes through receptor-mediated endocytosis. In order for the introduced substance to function in the cells, it must escape from the endosome and move to the cytoplasm or nucleus. The endosomal escape peptide of the present disclosure refers to a peptide having an endosomal escape ability, and it may help substances internalized into cells through endosomes more efficiently and quickly move to the nucleus or cytoplasm to meet and to act on a target gene.

The endosomal escape peptide may be an HA2 peptide, a CM18 peptide, or an S10 peptide. The HA2 peptide is a pH-sensitive amphiphilic peptide, and may include an amino acid sequence of SEQ ID NO: 13. The CM18 peptide and the S10 peptide are amphipathic a-helical peptides, which may form transmembrane channels in cell membranes or may disrupt membranes by a carpet mechanism, and may include an amino acid sequence of SEQ ID NO: 14 or SEQ ID NO: 15, respectively.

The endosomal escape peptide may be linked to the C-terminus of the nuclear localization sequence. Specifically, the endosomal escape peptide may be linked to the C-terminus of the nuclear localization sequence and may be linked to the N-terminus of the NKG2D ligand-derived protein.

Another aspect provides a polynucleotide encoding the fusion protein. The same as those described above are equally applied to the polynucleotide.

As used herein, the term “polynucleotide” refers to a polymer of deoxyribonucleotide or ribonucleotide that exists in a single-stranded or double-stranded form. The polynucleotide encompasses RNA genome sequences, DNA (gDNA and cDNA), and RNA sequences transcribed therefrom, and includes analogs of natural polynucleotides unless otherwise specified.

In the present disclosure, the nucleotide sequence encoding the fusion protein includes a nucleotide sequence encoding the amino acid described by each sequence number as well as a nucleotide sequence having 80% or more, specifically 90% or more, more specifically 95% or more, much more specifically 98% or more, the most specifically 99% or more homology to the sequence while encoding a protein exhibiting efficacy substantially identical or corresponding to that of each of the proteins. Further, it is apparent that as long as an amino acid sequence is a sequence having homology to the above sequence and has a biological activity substantially identical or corresponding to that of the fusion protein of the described sequence number, an amino acid sequence having deletion, modification, substitution, or addition in some sequence is also included in the scope of the present disclosure.

As used herein, the term “homology” refers to degree of similarity between nucleotide sequences encoding proteins or amino acid sequences constituting proteins. When the homology is sufficiently high, an expression product and a protein of the corresponding gene may have identical or similar activity. Further, homology may be expressed as a percentage according to a degree of matching with a given amino acid sequence or nucleotide sequence. In the present disclosure, a homology sequence having an activity which is identical or similar to the given amino acid sequence or nucleotide sequence is expressed as “% homology”. The homology sequence may be determined by, for example, a standard software, specifically, BLAST 2.0, which calculates parameters such as score, identity, similarity, etc., or by comparing the sequences in a Southern hybridization experiment under defined stringent conditions, and defining appropriate hybridization conditions are within the skill of the art, and may be determined by a method well known to those skilled in the art.

The fusion protein of the present disclosure may include a polynucleotide encoding the amino acid sequence of the above described sequence number, or a protein exhibiting 80% or more, 85% or more, 90% or more, 91% or more, 92% or more, 93% or more, 94% or more, 95% or more, 96% or more, 97% or more, 98% or more, 99% or more homology to the sequence, as long as it has a biological activity identical or corresponding to that of each protein.

Further, the polynucleotide encoding the fusion protein may undergo various modifications in the coding region without changing the amino acid sequence of the protein expressed from the coding region, due to codon degeneracy or in consideration of the codons preferred in an organism in which the protein is to be expressed. Therefore, the polynucleotide may include any polynucleotide sequence without limitation, as long as it encodes each protein.

Further, the polynucleotide includes not only the nucleotide sequence encoding the amino acid sequence of the fusion protein, but also a sequence complementary to the sequence. The complementary sequence includes not only a perfectly complementary sequence, but also a substantially complementary sequence, which refers to a sequence that may hybridize with the nucleotide sequence encoding the amino acid sequence of the fusion protein, for example, under stringent conditions known in the art.

The “stringent conditions” refer to conditions which allow a specific hybridization between polynucleotides. For example, the stringent conditions may include conditions under which genes having high homology, genes having 40% or more, specifically 90% or more, more specifically 95% or more, much more specifically 97% or more, and particularly specifically 99% or more homology hybridize with each other, while genes having homology lower than the above homology do not hybridize with each other, or may include ordinary washing conditions of Southern hybridization, i.e., washing once, specifically, twice or three times, at a salt concentration and a temperature corresponding to 60° C. 1×SSC, 0.1% SDS, specifically 60° C. 0.1×SSC, 0.1% SDS, and more specifically, 68° C. 0.1×SSC, 0.1% SDS.

Hybridization requires that two polynucleotides have complementary sequences, although mismatches between bases are possible depending on stringency of the hybridization. The term “complementary” is used to describe a relationship between nucleotide bases that may hybridize with each other. For example, with respect to DNA, adenosine is complementary to thymine, and cytosine is complementary to guanine. Therefore, the present disclosure may also include an isolated polynucleotide fragment complementary to the entire sequence as well as a polynucleotide sequence substantially similar thereto.

Specifically, a polynucleotide having homology may be detected using hybridization conditions including a hybridization step at a Tm value of 55° C. under the above-described conditions. Further, the Tm value may be 60° C., 63° C., or 65° C., but is not limited thereto, and may be appropriately controlled by those skilled in the art depending on the purpose thereof. The appropriate stringency for hybridizing polynucleotides depends on a length and degree of complementarity of the polynucleotides, and these variables are well known in the art.

Still another aspect provides an expression vector including the polynucleotide. The same as those described above are equally applied to the expression vector.

As used herein, the term “expression vector”, which is a recombinant vector introduced into an appropriate host cell to express a target protein, refers to a genetic construct including essential regulatory elements to which a gene insert is operably linked in such a manner as to be expressed. The term “operably linked” means a functional linkage between a nucleic acid expression regulatory sequence and a nucleic acid sequence encoding a target protein in such a manner as to allow general functions. The operable linkage to a recombinant vector may be prepared using a genetic recombinant technique well known in the art, and site-specific DNA cleavage and ligation may be carried out using enzymes generally known in the art.

An appropriate expression vector of the present disclosure may include a signal sequence for membrane targeting or secretion in addition to expression control elements such as a promoter, a start codon, a stop codon, a polyadenylation signal, and an enhancer. Start codon and stop codon are generally considered as a part of a nucleotide sequence encoding an immunogenic target protein, and need to have actions in an individual when a gene construct is administered and be in frame with a coding sequence. A general promoter may be constitutive or inducible. In prokaryotic cells, the promoter includes lac, tac, T3, and T7 promoters. In eukaryotic cells, the promoter includes a monkey virus 40 (SV40) promoter, a mouse mammary tumor virus (MMTV) promoter, a human immunodeficiency virus (HIV), for example, a long terminal repeat (LTR) promoter of HIV, molonivirus, cytomegalovirus (CMV), Epstein barr virus (EBV), and rous sarcoma virus (RSV) promoters, as well as a β-actin promoter, and human hemoglobin, human muscle creatine, and human metallothionein-derived promoters, but is not limited thereto.

Further, the expression vector may include a selective marker for selecting host cells including the vector. The selective marker is to select cells transformed by the vector, and markers giving selectable phenotypes such as drug resistance, auxotrophy, resistance to a cytotoxic agent, or expression of a surface protein may be used. Since only the cells expressing the selective marker survive in an environment treated with a selective agent, it is possible to select the transformed cells. Further, when the vector is a replicable expression vector, the vector may include a replication origin which is a specific nucleic acid sequence in which replication is initiated.

As the recombinant expression vector for inserting a foreign gene, various types of vectors including plasmids, viruses, cosmids, etc. may be used. The type of recombinant vector is not particularly limited as long as the recombinant vector functions to express a desired gene and to produce a desired protein in various types of host cells of prokaryotes and eukaryotes. Specifically, the recombinant vector is a vector capable of mass-producing a foreign protein having a similar form to a natural state while retaining a promoter having strong activity and a strong expression ability.

To express the protein of the present disclosure, a wide variety of expression host/vector combinations may be employed. Expression vectors suitable for eukaryotic hosts may include, but are not limited to, expression control sequences derived from SV40, bovine papillomavirus, adenovirus, adeno-associated virus, cytomegalovirus, retrovirus, etc. Expression vectors that may be used in bacterial hosts may include, but are not limited to, bacterial plasmids obtained from Escherichia coli, including pET21a, pET, pRSET, pBluescript, pGEX2T, pUC vector, col El, pCR1, pBR322, pMB9, or derivatives thereof, plasmids with a broad host range such as RP4, phage DNA exemplified by phage lambda derivatives such as λgt10, λgt11, NM989, etc., and other DNA phages such as M13 and filamentous single-stranded DNA phage, etc. 2° C. plasmid or derivatives thereof may be used for yeast cells, and pVL941, etc. may be used for insect cells.

The cell, for example, eukaryotic cell may be a cell of yeast, fungus, protozoa, plant, higher plant, insect, or amphibian, or a mammalian cell such as CHO, HeLa, HEK293, and COS-1, for example, cultured cells (in vitro), transplanted cells (graft cells), and primary culture cells (in vitro and ex vivo), and in vivo cells which are commonly used in the art, and may also be cells of mammals including humans (mammalian cells). Further, the organism may be a yeast, a fungus, a protozoa, a plant, a higher plant, an insect, an amphibian, or a mammal.

Still another aspect provides a gene editing complex including the fusion protein including the Cas protein and the NKG2D ligand-derived protein; and guide RNA. The same as those described above are equally applied to the gene editing complex.

The fusion protein enables complex formation by an electrostatic interaction with guide RNA, without being limited to a particular theory. Therefore, the complex may be self-assembled by the fusion protein to form a complex with guide RNA, and the complex may efficiently achieve co-delivery to the nucleus.

The guide RNA may be a dual RNA including CRISPR RNA (crRNA) and transactivating crRNA (tracrRNA), or a single-chain guide RNA (sgRNA) including a portion of the crRNA and tracrRNA and hybridizing with the target DNA.

As used herein, the terms “guide RNA”, “chimeric RNA”, “chimeric guide RNA”, “single guide RNA” and “synthetic guide RNA” are used interchangeably, and refers to a polynucleotide sequence including a guide sequence, a tracr sequence, and/or a tracr mate sequence. The term “guide sequence” refers to about 20 bp sequence within the guide RNA that specifies a target site, and may be used interchangeably with the terms “guide” or “spacer”. The term “tracr mate sequence” may also be used interchangeably with the term “direct repeat(s)”. The guide RNA may consist of two RNAs, i.e., CRISPR RNA (crRNA) and transactivating crRNA (tracrRNA), or may be a single-chain RNA (sgRNA) including a portion of the crRNA and tracrRNA and hybridizing with the target DNA.

In general, a guide sequence is any polynucleotide sequence having sufficient complementarity with a target polynucleotide sequence to hybridize with the target DNA sequence and to direct sequence-specific binding of the CRISPR complex to the target DNA sequence. In some embodiments, the degree of complementarity between the guide sequence and its corresponding target sequence, when optimally aligned using a suitable alignment algorithm, is about 50%, 60%, 75%, 80%, 85%, 90%, 95%, 97.5%, 99% or more. Optimal alignment may be determined with the use of any suitable algorithm for aligning sequences, non-limiting example of which includes the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies), ELAND (Illumina, San Diego, Calif., USA), SOAP (available at soap.genomics.org.cn), and Maq (available at maq.sourceforge.net). In some embodiments, the guide sequence is about 5, 10, 11, 12, 13, 14, 15, 16, 17, 18, 19, 20, 21, 22, 23, 24, 25, 26, 27, 28, 29, 30, 35, 40, 45, 50, 75 or more nucleotides in length. In some embodiments, the guide sequence is about 75, 50, 45, 40, 35, 30, 25, 20, 15, 12 or less nucleotides in length. The ability of the guide sequence to direct sequence-specific binding of the CRISPR complex to a target sequence may be assessed by any suitable assay. For example, components of the CRISPR system sufficient to form the CRISPR complex, including the guide sequence to be tested, may be provided to a host cell having the corresponding target sequence, for example, by transfection with vectors encoding the components of the CRISPR sequence, followed by an assessment of preferential cleavage within the target sequence, for example, by Surveyor assay as described herein. Similarly, cleavage of a target polynucleotide sequence may be evaluated in a test tube by providing the target sequence, components of the CRISPR complex, including the guide sequence to be tested and a control guide sequence different from the test guide sequence, and comparing rate of binding or cleavage at the target sequence between the test and control guide sequence reactions. Other assays are possible, and will occur to those skilled in the art.

The guide sequence may be selected to target any target DNA sequence. In some embodiments, the target DNA sequence is a sequence within a genome of a cell. Exemplary target sequences include those unique in the target genome. For example, with respect to Streptococcus pyogenes Cas9, a unique target sequence in a genome may include a Cas9 target site of a form of MMMMMMMMNNNNNNNNNNNNXGG, where NNNNNNNNNNNNXGG (N is A, G, T, or C; and X may be anything) has a single occurrence in the genome. A unique target sequence in a genome may include a Streptococcus pyogenes Cas9 target site of a form MMMMMMMMMNNNNNNNNNNNXGG, where NNNNNNNNNNNXGG (N is A, G, T, or C; and X may be anything) has a single occurrence in the genome. With respect to Streptococcus thermophilus CRISPR1 Cas9, a unique target sequence in a genome may include a Cas9 target site of a form MMMMMMMMNNNNNNNNNNNNXXAGAAW, where NNNNNNNNNNNNXXAGAAW (N is A, G, T, or C; X may be anything; and W is A or T) has a single occurrence in the genome. A unique target sequence in a genome may include a Streptococcus thermophilus CRISPR1 Cas9 target site of a form MMMMMMMMMNNNNNNNNNNNXXAGAAW, where NNNNNNNNNNNXXAGAAW (N is A, G, T, or C; X may be anything; and W is A or T) has a single occurrence in the genome. With respect to Streptococcus pyogenes Cas9, a unique target sequence in a genome may include a Cas9 target site of a form MMMMMMMMNNNNNNNNNNNNXGGXG, where NNNNNNNNNNNNXGGXG (N is A, G, T, or C; and X may be anything) has a single occurrence in the genome. A unique target sequence in a genome may include a Streptococcus pyogenes Cas9 target site of a form MMMMMMMMMNNNNNNNNNNNXGGXG, where NNNNNNNNNNNXGGXG (N is A, G, T, or C; and X may be anything) has a single occurrence in the genome. In each of these sequences, “M” may be A, G, T, or C.

Since the gene editing complex may target one or more DNAs or genes and may edit multiple genes, it may edit multiple gene loci at the same time. The gene editing refers to manipulating a gene, and may specifically include deletion such as gene knock-out, knock-down, etc., gene insertion (knock-in), gene correction, gene expression control, chromosome rearrangement, etc.

The gene knock-out may refer to regulation of gene activity, for example, inactivation by deletion, substitution of all or part of the gene (e.g., one or more nucleotides), and/or insertion of one or more nucleotides. The gene inactivation refers to suppression or downregulation of gene expression or a modification to encode a protein that has lost its original function. Further, gene regulation may refer to a change in gene function, resulting from a structural modification of a protein obtained by deletion of exon sites due to simultaneous targeting of both intron sites surrounding one or more exons of a target gene, expression of a dominant negative form of a protein, expression of a competitive inhibitor secreted in a soluble form, etc.

The gene insertion (knock-in) means inserting a foreign base sequence that exists in another species or does not exist originally in an organism into a genome of the corresponding organism or a DNA sequence derived from the corresponding organism using a genetic recombination technology.

The complex may further include an endosomal escape peptide, and specifically, the endosomal escape peptide may be included in the fusion protein or included as a separate protein in the complex.

The complex may further include donor DNA for gene knock-in.

The gene editing complex may be to manipulate a target gene or a target DNA of an NKG2D receptor-expressing cell or a natural killer cell.

As used herein, the term “natural killer cell” is an important lymphocyte cell responsible for innate immunity, and accounts for 5% to 10% of all lymphocytes, and matures in the liver or bone marrow, unlike T cells. It is known that natural killer cells are able to differentiate normal cells from abnormal cells by expressing various innate immune receptors on the cell surface, and when target cells such as virus-infected cells, tumor cells, etc. are recognized, they may be immediately attacked and removed. Natural killer cells that recognize abnormal cells secrete perforin to create pores in the cell membrane of the target cells, and secretes granzyme into the cell membrane to disintegrate the cytoplasm, causing apoptosis, or injects water and salts into the cells, causing cell necrosis. In addition, as an indirect method, natural killer cells secrete cytokines to activate cytotoxic T cells and B cells. Both the number and high activity of natural killer cells are known to be very important measures for these immune effects mediated by natural killer cells.

The complex may be specifically delivered to NKG2D receptor-expressing cells or natural killer cells without a carrier to manipulate genes of natural killer cells. Thus, when the complex is used, the cancer treatment effect of natural killer cells may be improved.

Still another aspect provides a composition for editing a target DNA or a target gene specifically to NKG2D receptor-expressing cells or natural killer cells, the composition including the gene editing complex. The same as those described above are equally applied to the composition.

The gene editing complex included in the composition is characterized in that it is carrier-free, and does not require a separate carrier for transformation, such as lipofectamine, etc. In one aspect, to increase transformation efficiency of the composition, a transformation carrier may be further included.

In addition, the composition may effectively edit a target DNA of a eukaryotic cell ex vivo or in vivo. For example, the cell may be cultured cells (in vitro), transplanted cells (graft cells), and primary culture cells (in vitro and ex vivo), and in vivo cells which are commonly used in the art, or cells of organisms or cells of mammals including humans (mammalian cells).

Still another aspect provides a method of editing a target DNA or a target gene specifically to NKG2D receptor-expressing cells or natural killer cells, comprising treating to the NKG2D receptor-expressing cells or natural killer cells the gene editing complex. The same as those described above are equally applied to the method.

The gene editing complex is characterized in that it is carrier-free, and does not require a separate carrier for transformation, such as lipofectamine. In one aspect, to increase transformation efficiency of the composition, a transformation carrier may be further included.

In addition, the method may effectively edit a target DNA of a eukaryotic cell ex vivo or in vivo. For example, the cell may be cultured cells (in vitro), transplanted cells (graft cells), and primary culture cells (in vitro and ex vivo), and in vivo cells which are commonly used in the art, or cells of organisms or cells of mammals including humans (mammalian cells).

Still another aspect provides a pharmaceutical composition for treating or preventing cancer, the pharmaceutical composition, comprising the gene editing complex of the present disclosure as an active ingredient. The same as those described above are equally applied to the composition.

As used herein, the term “cancer” refers to a tumor abnormally grown by autonomous overgrowth of body tissues, or a tumor-forming disease. The cancer may be specifically liver cancer, lung cancer, pancreatic cancer, non-small cell lung cancer, colon cancer, bone cancer, skin cancer, head or neck cancer, skin or intraocular melanoma, uterine cancer, ovarian cancer, rectal cancer, gastric cancer, perianal cancer, colon cancer, breast cancer, fallopian tube carcinoma, uterine endometrial carcinoma, cervical carcinoma, vaginal carcinoma, vulva carcinoma, Hodgkin's disease, esophageal cancer, small intestine cancer, endocrine cancer, thyroid cancer, parathyroid cancer, adrenal gland cancer, soft tissue sarcoma, urethral cancer, penile cancer, prostate cancer, chronic or acute leukemia, lymphocytic lymphoma, bladder cancer, kidney or ureter cancer, renal cell carcinoma, renal pelvic carcinoma, central nervous system (CNS) tumor, primary CNS lymphoma, spinal tumor, brainstem glioma, or pituitary adenoma.

The term “treating”, as used herein, means all actions that improve or beneficially change symptoms of a cancer disease via administration of the composition of the present disclosure.

The term “preventing”, as used herein, means all actions that inhibit or delay a cancer disease or the onset of a disease via administration of the composition of the present disclosure.

The composition effectively performs gene editing, such as manipulating a target DNA or a target gene specifically to NKG2D receptor-expressing cells or natural killer cells, thereby inducing natural killer cells with improved effects of preventing or treating cancer, such as cancer cell-killing effects, etc. Accordingly, the composition may exhibit cancer therapeutic effects.

The pharmaceutical composition may include a pharmaceutically acceptable carrier. The “pharmaceutically acceptable carrier” may refer to a carrier or diluent that does not cause significant irritation to an organism and does not abrogate biological activity and properties of the administered compound. Here, “pharmaceutically acceptable” means that an object to be applied (prescribed) does not have toxicity beyond adaptable without inhibiting the activity of the active ingredient.

Any kind of carrier that may be used in the present disclosure may be used, as long as it is commonly used in the art and is pharmaceutically acceptable. Non-limiting examples of the carrier may include a saline solution, sterile water, Ringer's solution, buffered saline, an albumin injectable solution, a dextrose solution, a maltodextrin solution, glycerol, ethanol, etc. These may be used alone or in a mixture of two or more thereof. The pharmaceutical composition may be prepared in an oral dosage form or a parenteral dosage form according to an administration route by a common method known in the art, by including a pharmaceutically acceptable carrier in addition to the active ingredient.

The pharmaceutical composition may be formulated into an oral dosage form such as powder, granules, tablets, capsules, suspensions, emulsions, syrups, aerosols, etc., external preparations, suppositories, or sterile injectable solutions according to common methods, respectively. When the pharmaceutical composition is formulated, it may be prepared by adding diluents or excipients such as fillers, extenders, binders, wetting agents, disintegrants, surfactants, etc., which are commonly used.

When the pharmaceutical composition is prepared in an oral dosage form, it may be prepared into a formulation, such as powders, granules, tablets, pills, sugar-coated tablets, capsules, liquids, gels, syrups, suspensions, wafers, etc., together with an appropriate carrier, in accordance with methods known in the art. Here, examples of the pharmaceutically acceptable carrier may include saccharides such as lactose, glucose, sucrose, dextrose, sorbitol, mannitol, xylitol, etc., starch such as corn starch, potato starch, wheat starch, etc., celluloses such as cellulose, methyl cellulose, ethyl cellulose, sodium carboxymethyl cellulose, hydroxypropylmethyl cellulose, etc., polyvinyl pyrrolidone, water, methylhydroxybenzoate, propylhydroxybenzoate, magnesium stearate, mineral oil, malt, gelatin, talc, polyol, vegetable oil, etc. When formulated, formulation may be performed by including diluents and/or excipients, such as fillers, extenders, binders, wetting agents, disintegrants, surfactants, etc., as needed.

When the pharmaceutical composition is prepared in a parenteral dosage form, it may be formulated in the form of injectable formulations, transdermal delivery systems, nasal inhalers, and suppositories, together with an appropriate carrier, in accordance with methods known in the art. An appropriate carrier for injectable formulations may include sterile water, ethanol, polyols such as glycerol or propylene glycol, or a mixture thereof, and preferably, isotonic solutions such as Ringer's solution, phosphate-buffered saline (PBS) containing triethanolamine, sterile water for injection, 5% dextrose, etc. When formulated into a transdermal delivery system, the pharmaceutical composition may be formulated into ointments, creams, lotions, gels, liquids for external use, pastes, liniments, aerosols, etc. For nasal inhalers, the pharmaceutical composition may be formulated in the form of an aerosol spray using an appropriate propellant such as dichlorofluoromethane, trichlorofluoromethane, dichlorotetrafluoroethane, carbon dioxide, etc. When formulated into suppositories, Witepsol, Tween 61, polyethylene glycols, cacao butter, laurin butter, polyoxyethylene sorbitan fatty acid esters, polyoxyethylene stearates, sorbitan fatty acid esters, etc. may be used as a base thereof.

The pharmaceutical composition may be administered in a pharmaceutically effective amount, and the term “pharmaceutically effective amount” means an amount which is sufficient to treat or prevent diseases at a reasonable benefit/risk ratio applicable to any medical treatment or prevention. The effective dosage level may be determined depending on factors including severity of a disease, activity of a drug, a patient's age, body weight, health, sex, sensitivity to a drug, administration time, administration route, and excretion rate of the composition of the present disclosure, duration of treatment, drugs used in combination or concurrently with the composition of the present disclosure, and other factors well known in the medical field. The pharmaceutical composition may be administered alone or in combination with components known to exhibit therapeutic effects on cancer. It is important to administer the composition in a minimum amount that may exhibit a maximum effect without causing side effects, considering all the above-described factors.

The dosage of the pharmaceutical composition may be determined by those skilled in the art in consideration of the purpose of use, severity of a disease, a patient's age, body weight, sex, history, a type of a substance used as an active ingredient, etc. For example, the pharmaceutical composition of the present disclosure may be generally administered in a dosage of about 0.1 ng to about 1,000 mg/kg, preferably, about 1 ng to about 100 mg/kg for an adult, and administration frequency of the composition of the present disclosure may be, but is not particularly limited to, once per day or in several divided doses per day. The dosage or administration frequency does not limit the scope of the present disclosure in any aspect.

Still another aspect provides a method for treating or preventing cancer, comprising administering to a subject the pharmaceutical composition for treating or preventing cancer. The same as those described above are equally applied to the method.

As used herein, the term “subject” may include, without limitation, mammals including rats, livestock, humans, etc., birds, reptiles, farmed fish, etc. developing or at risk of developing cancer.

The pharmaceutical composition may be administered in a single or multiple dose of the pharmaceutically effective amount. In this regard, the composition may be administered after being formulated into the form of a liquid, powder, aerosol, injectable formulation, infusion solution (Ringer's solution), capsule, pill, tablet, suppository, or patch. An administration route of the pharmaceutical composition for preventing or treating cancer may be any general route, as long as the pharmaceutical composition may reach a target tissue.

The pharmaceutical composition may be, but is not particularly limited to, administered via intraperitoneal administration, intravenous administration, intramuscular administration, subcutaneous administration, intradermal administration, transdermal patch administration, oral administration, intranasal administration, intrapulmonary administration, rectal administration, etc. according to the purpose. However, when administered orally, the pharmaceutical composition may be administered in an unformulated form. Since the active ingredient of the pharmaceutical composition may be denatured or decomposed by gastric acid, an oral composition may be orally administered after coating the active ingredient, or in a formulated form to protect the active ingredient from decomposition in the stomach, or in a form of an oral patch. In addition, the composition may be administered by any apparatus capable of transferring the active ingredient to target cells.

### BRIEF DESCRIPTION OF THE DRAWINGS

The above and other aspects, features, and advantages of certain embodiments of the disclosure will be more apparent from the following description taken in conjunction with the accompanying drawings, in which:

FIGS. 1A and 1B shows results of examining TGFBR2 expression levels in NK cell lines;

FIG. 2 shows TGFBR2 knock-out efficiencies of different designed sgRNAs;

FIG. 3 shows results of examining NKG2D expression levels in NK cell lines;

FIG. 4 shows a schematic illustration of a Cas9 fusion protein designed in the present disclosure;

FIG. 5 shows a DNA vector expressing the Cas9 fusion protein designed in the present disclosure;

FIGS. 6A and 6B shows results of purifying the Cas9 fusion protein designed in the present disclosure;

FIGS. 7A and 7B shows efficiency of internalization of the Cas9 fusion protein designed in the present disclosure into NK cells;

FIG. 8 shows efficiency of internalization of a Cas9-RAE1 fusion protein designed in the present disclosure into NK cells;

FIGS. 9A and 9B shows gene knock-out efficiency of a gene editing complex including the Cas9 fusion protein designed in the present disclosure;

FIGS. 10A to 10D shows gene knock-out efficiency of a gene editing complex including the Cas9-RAE1 fusion protein designed in the present disclosure;

FIGS. 11A and 11B shows a donor vector designed for gene knock-in; and

FIGS. 12A and 12B shows gene knock-in efficiency of a gene editing complex including the Cas9 fusion protein designed in the present disclosure.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the present description. As used herein, the term “and/or” includes any and all combinations of one or more of the associated listed items. Expressions such as “at least one of,” when preceding a list of elements, modify the entire list of elements and do not modify the individual elements of the list.

Hereinafter, the present disclosure will be described in more detail with reference to exemplary embodiments. However, these exemplary embodiments are only for illustrating, and the scope of the present disclosure is not limited to these exemplary embodiments.

### Example 1: Selection of Target Knock-out Gene in Natural Killer Cells

The present disclosure relates to a fusion protein capable of performing gene editing, such as gene knock-out, etc., without a carrier, and a gene editing complex including the same. In particular, natural killer cell-specific gene editing may be performed. Accordingly, to examine whether gene editing may be efficiently performed in natural killer cells by using the fusion protein of the present disclosure, TGFBR2 known to be expressed in natural killer cells was selected as a representative target gene, and sgRNA targeting TGFBR2 was designed and prepared through the following experiments.

### 1-1. Examination of Expression Levels of TGFBR2 in Natural Killer Cells

To examine whether transforming growth factor-beta receptor type 2 (TGFBR2) was expressed in natural killer cells, the following experiment was performed.

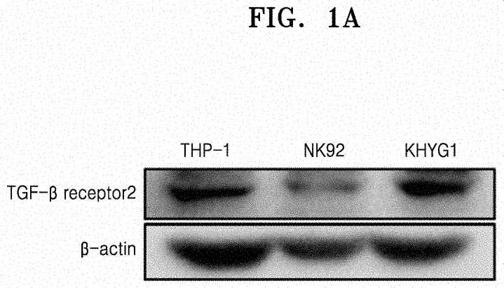

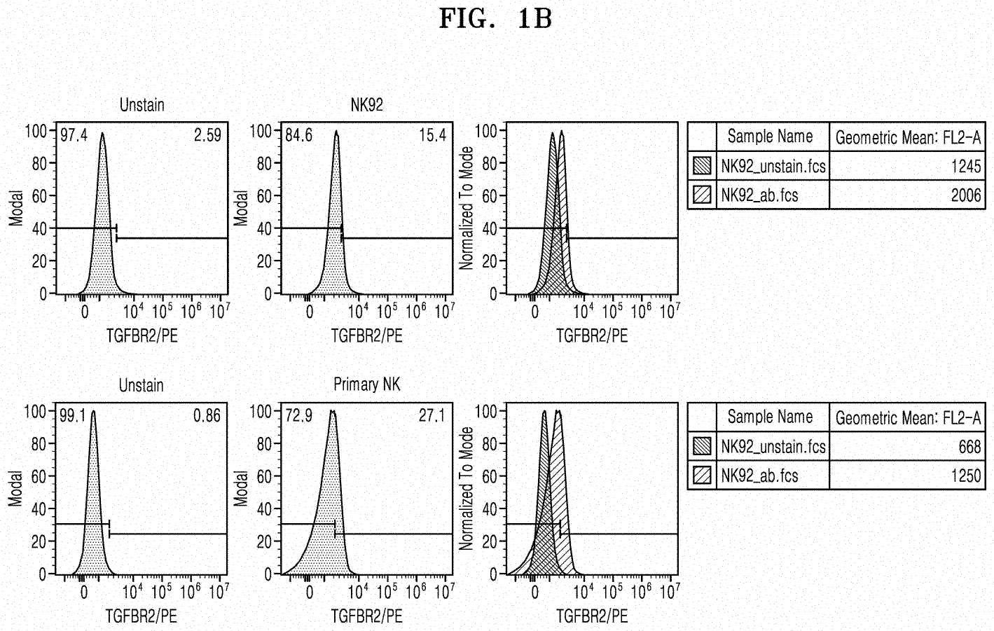

First, to examine the expression using Western blotting, RIPA buffer (SIGMA) was added to each 2×10⁵ cells of THP1 (monocyte cell line), NK92 and KHYG1 (NK cell lines) to obtain each cell lysate, and then sampling was performed to prepare samples for Western blotting. The samples were electrophoresed on SDS-PAGE, and transferred onto a nitrocellulose membrane, followed by blocking with skim milk. Next, the membrane was incubated with a TGFbR2 primary antibody (1:1000, cell signaling technology) diluted at a ratio of 1:1000 and a β-actin (SantaCruz) primary antibody diluted at a ratio of 1:5000 at 4° C. for one day. After washing, the membrane was incubated with a horseradish peroxidase-conjugated secondary antibody (1:5000) capable of binding to the primary antibody for 2 hours at room temperature. Then, the antibody was visualized by chemiluminescence using a Western ECL substrate (Thermo scientific), and the obtained luminescent images were analyzed by Chemidoc (biorad). As a result, TGFBR2 expression was observed in all of THP1, NK92, and KHYG1 cells (FIG. 1A).

Next, in order to examine expression of TGFbR2 located on the cell membranes in NK92 and primary NK cells, the cells were stained with a TGFbR2-PE (1:100, R&D) antibody, followed by flow cytometry. As a result, TGFBR2 expression was observed on the surface of all of the cells (FIG. 1B).

Based on the above results, it was confirmed that TGFBR2 was expressed on the cell membrane of the NK cell lines, indicating that a gene encoding TGFBR2 may be selected as a knock-out target.

### 1-2. Design of sgRNA for TGFBR2 Knock-Out

To design sgRNA for TGFBR2 knock-out using a CRISPR gene editing technology, the following experiment was performed.

First, to select an sgRNA sequence specific to a target sequence for TGFbR2 gene editing, several sgRNA sequences were prepared in the form of RNA (Table 1), and 4 ul of each sgRNA and Cas9 (spCas9-NLS WT, 1 ug/ml, 4 ul) were reacted in phosphate buffered saline (PBS) at room temperature for 15 minutes to prepare Cas9 ribonucleoproteins (Cas9 RNPs).

TABLE 1

| | | | SEQ |
|---|---|---|---|
| | | | ID |
| Sequence name | Sequence | Location | NO: |
| hTGFBR2_crRNA#7 | ggccgctgcacatcgtcctg | exon1 | 16 |
| hTGFBR2_crRNA#8 | cccaccgcacgttcagaagt | exon1 | 17 |
| hTGFBR2_crRNA#9 | ccgacttctgaacgtgcggt | exon1 | 18 |
| hTGFBR2_crRNA#2 | cccctaccatgactttattc | exon4 | 19 |
| hTGFBR2_crRNA#10 | ccgcgtcttgccggtttccc | exon4 | 20 |
| hTGFBR2_crRNA# | CCTGAGCAGCCCCCGACCCA | exon1 | 21 |
| ref | | | |

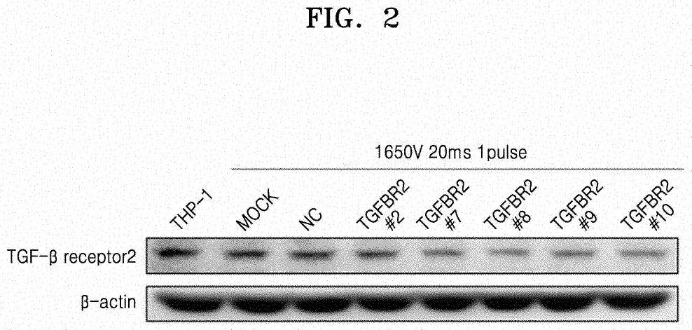

Each Cas9 RNP was delivered to 1×10⁶ THP1, which is a monocyte cell line, using an electroporation device (Neon electroporation) under conditions of 1650 V, 20 ms, and 1 pulse. After electroporation and incubation for 48 hours, Western blotting was performed in the same manner as above to compare TGFbR2 expression levels. As a result, it was confirmed that #7 and #8, among the prepared sgRNAs, had excellent TGFbR2 inhibition efficiency (FIG. 2).

The above results indicate that the sgRNA sequences prepared above may effectively knock-out TGFBR2. In the following exemplary embodiment, the sgRNA sequences prepared above were used to examine the gene knock-out efficiency of the gene editing system of the present disclosure.

### Example 2: Construction of Natural Killer Cell-Specific Gene Editing System

The gene editing system of the present disclosure is charactered in that it may be specifically introduced into natural killer cells without a separate carrier and may operate therein. To this end, the fusion protein of the present disclosure was allowed to be specifically introduced into natural killer cells without a carrier by including a ligand binding to a receptor expressed on the surface of natural killer cells. Therefore, the receptor expressed on the surface of natural killer cells was identified and the type of the ligands binding thereto was specified to construct a system, as follows.

### 2-1. Selection of Protein Expressed Specifically to Natural Killer Cells

To construct a system for delivering the gene editing complex specifically to NK cells without a carrier, a protein expressed on the surface of NK cells was first selected.

In detail, among various receptors expressed on the surface of NK cells, NKG2D was selected as a candidate targeting receptor. The NKG2D is known to be capable of inducing ubiquitin-dependent endocytosis by binding to a ligand thereof, and thus it is expected that a fusion protein targeting this may be effectively internalized into cells by binding with NKG2D.

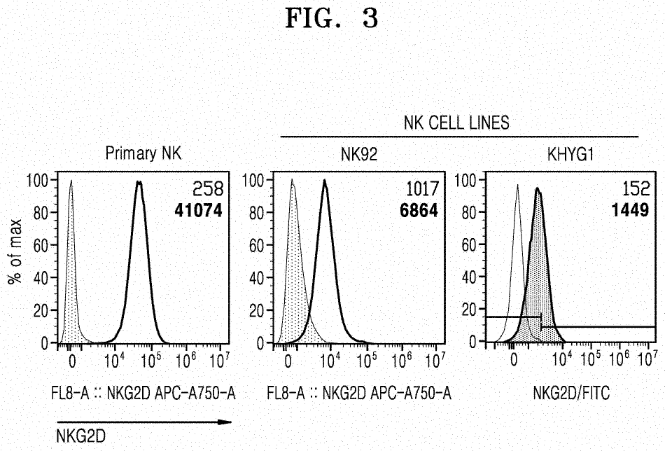

Next, to examine whether NKG2D was expressed on the cell membranes of NK92 and KHYG1 which are NK cell lines, and primary NK cells, 2×10⁵ cells of each cell were stained with NKG2D-APC (Biolegend, 1:100) or NKG2D-FITC (Biolegend, 1:100) antibody, and NKG2D expression on the cell surface was analyzed by flow cytometry. As a result, it was confirmed that NKG2D was expressed on the surface of all the cells (FIG. 3), and a fusion protein was designed using NKG2D of NK cells as a target.

### 2-2. Design and Preparation of Fusion Protein

To construct a natural killer cell-specific gene editing system, the following Cas fusion protein was designed.

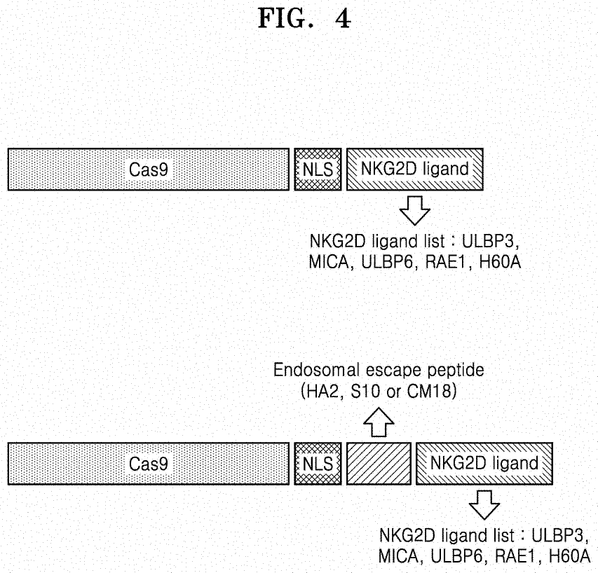

In detail, Cas9 protein was used as a Cas protein, and a nuclear localization sequence (nuclear localization signal, NLS) was included to transport the gene editing system into the cell nucleus. Next, to specifically target NK cells, a NKG2D ligand capable of binding to NKG2D expressed on the surface of NK cells was included. As the NKG2D ligand, UL16 binding protein 3 (ULBP3), MHC Class I Polypeptide-Related Sequence A (MICA), ULBP6, Retinoic acid early-inducible protein 1-beta (RAE1), and histocompatibility antigen 60a (H60A) were selected and included, respectively. Next, to effectively deliver, to the nucleus, the gene editing system that binds to NKG2D to be internalized into NK cells through endocytosis, an endosomal escape peptide was included. As the endosomal escape peptide, HA2, S10 or CM18 peptide was used. As two types of fusion proteins designed through the above process, a fusion protein including Cas9, NLS, and NKG2D ligand, and a fusion protein including Cas9, NLS, endosomal escape peptide, and NKG2D ligand were designed, respectively (FIG. 4).

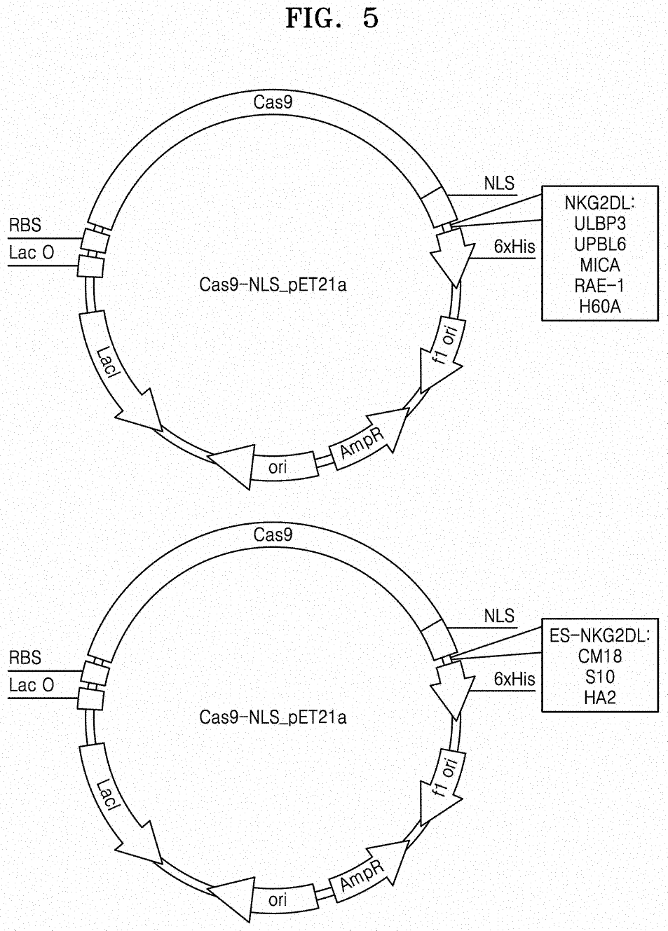

DNA expression vectors each for preparing the fusion proteins designed above were designed as shown in FIG. 5. In detail, for amplification of the NKG2D ligand gene, nucleotide sequences were used, each encoding ULBP3 protein (SEQ ID NO: 1) having an amino acid sequence from aspartate (Asp) at position 30 to arginine (Arg) at position 207, ULBP6 protein (SEQ ID NO: 3) having an amino acid sequence from aspartate (Asp) at position 29 to serine (Ser) at position 203, MICA protein (SEQ ID NO: 5) having an amino acid sequence from methionine (Met) at position 1 to serine (Ser) at position 297, RAE1 protein (SEQ ID NO: 7) having an amino acid sequence from aspartate (Asp) at position 31 to lysine (Lys) at position 204, and H60a protein (SEQ ID NO: 9) having an amino acid sequence from leucine (Leu) at position 20 to leucine (Leu) at position 214.

PCR reaction was performed with a gene amplifier using primers (Table 2) which were synthesized for amplification of each gene fragment.

TABLE 2

| Template | | | Sequence |
|---|---|---|---|
| DNA | Direction | Primer sequence | No. |
| ULBP3 | Forward | 5′-CCCGGTCTCCTAAGGACGCTCACTCTCTCTGGT-3′ | 22 |
| | Reverse | 5′-CCCGGTCTCCCCATTTCCAGCCTCTTCTTCCTGT-3 | 23 |
| ULBP6 | Forward | 5′-CCCGGTCTCCTAAGGACCCTCACTCTCTTTGCTA-3 | 24 |
| | Reverse | 5′-CCCGGTCTCCCCATGCTGTCCATGCCCATCAAG-3 | 25 |
| MICA | Forward | 5′-CCCGGTCTCCTAAGATGGGGCTGGGCCCGGT-3 | 26 |
| | Reverse | 5′-CCCGGTCTCCCCATAGAGGGCACAGGGTGAGTG-3 | 27 |
| RAE1 | Forward | 5′-CCCGGTCTCCTAAGGACGCCCATAGTTTACGCTG-3 | 28 |
| | Reverse | 5′-CCCGGTCTCCCCATTTTCTCTTTCGATTGTTTCAGAA-3 | 29 |
| H60a | Forward | 5′-CCCGGTCTCCTAAGCTTTTGTCGTACCTTGGCAC-3 | 30 |
| | Reverse | 5′-CCCGGTCTCCCCATGAGGCCTTGATTATCACTGTT-3 | 31 |
| Cas9- | Forward | 5′-CCCGGTCTCCATGGATAAGCATCACCACCAC-3 | 32 |
| NLS_pET21a | Reverse | 5′-CCCGGTCTCCCTTATCCATCACCTTCCTCTT-3 | 33 |

The forward primers and reverse primers include a base sequence (5′-GGTCTC-3′) corresponding to the Bsal restriction enzyme recognition site. PCR was performed using ULBP3, ULBP6, MICA, RAE1, and H60a as templates as follows. Distilled water was added to a mixed solution of 0.5 μl of each template DNA at a concentration of about 100 ng/μl, 1 μl of dNTP at a concentration of 10 mM, 5 μl of 5-fold concentrated PCR buffer (Thermofisher, USA), 1.5 μl of 100% dimethyl sulfoxide (DMSO), each 0.5 μl of forward and reverse primers at a concentration of 100 pmol/μl, and 0.5 μl of Phusion DNA polymerase (2 U/μl, Thermofisher, USA) to prepare a total of 50 μl of a reaction solution. After preheating the reaction solution at 98° C. for 30 seconds using a gene amplifier, the reaction at 98° C. for 10 seconds, at 60° C. for 30 seconds, and at 72° C. for 15 seconds was repeated 30 times. The final amplification step was performed at 72° C. for 5 minutes. The reaction solution was separated on a 0.8% agarose gel by electrophoresis to elute genes.

Each fragment of NKG2D ligand DNA obtained in the above step and pET21a vector fragment containing Cas9 were put in each reaction tube at a molecular ratio of 2:1, and then 2 μl of 10-fold concentrated ligation reaction buffer, each 1 μl of T4 DNA ligase (400 U/μl, NEB, USA) and Bsal restriction enzyme (10 U/μl, NEB, USA) were added, and distilled water was added to a total of 20 μl. This reaction solution was allowed to react at 37° C. for 1 hour, and the enzymes in the reaction tube were inactivated at 65° C. for 5 minutes. The reaction solution was added to transform E. coli DH5a (Thermofisher, USA) competent cells, and plated on an LB solid medium containing 100 μg/ml ampicillin to select E. coli transformants. The plasmid was extracted from this E. coli, and an expression vector Cas9-NLS-NKG2DL_pET21a was identified by nucleotide sequence analysis, in which each NKG2D ligand DNA fragment (ULBP3, ULBP6, MICA, RAE1, H60a) was linked to the pET21a plasmid containing Cas9.

To additionally express an endosomal escape sequence, PCR reaction was performed with a gene amplifier using the primers synthesized for amplification of each gene fragment, in order to bind CM18, HA2, and S10 DNA fragments to the previously cloned Cas9-NLS-NKG2DL_pET21a. Then, cloning was performed in the similar manner as above.

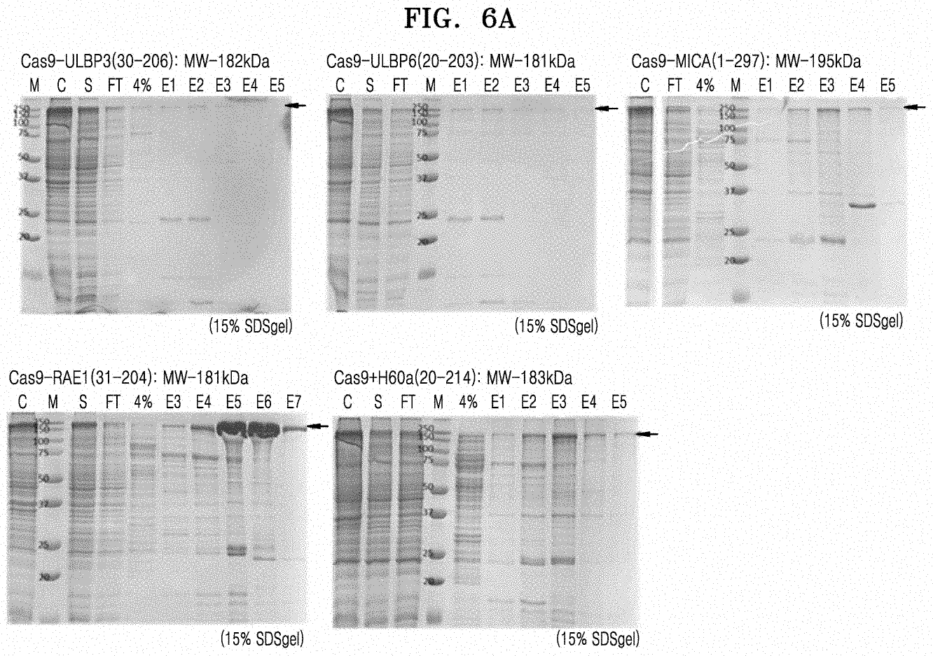

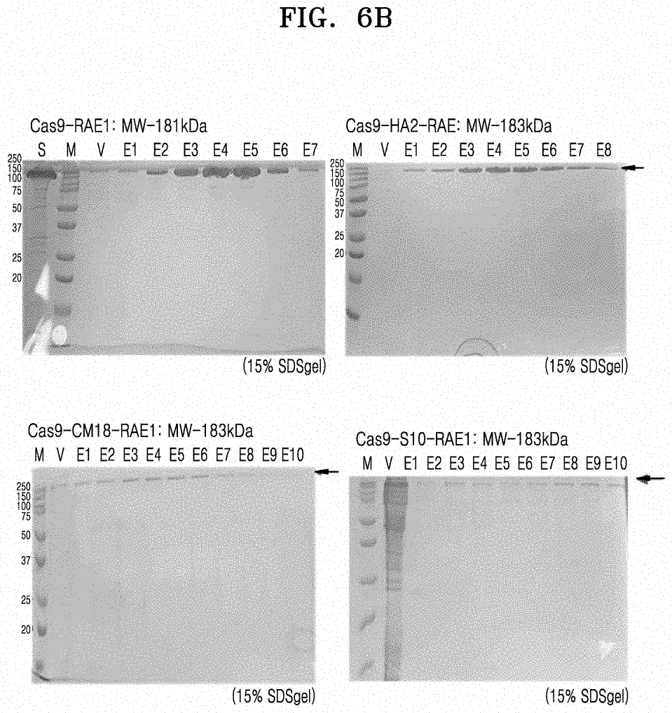

To prepare the fusion protein designed above, specifically, the DNA plasmid encoding each fusion protein prepared above was transformed into E. coli BL21 cells, and incubated on an ampicillin (100 μg/ml)-containing Luria-Bertani (LB) agar plate at 37° C. overnight. To induce fusion protein expression, transfected BL21 cells were incubated in 400 ml of LB-ampicillin medium containing 0.2 mM isopropyl-β-D-thiogalactopyranoside (IPTG) at 18° C. overnight. Cells were recovered by ultracentrifugation, and lysed by sonication in a lysis buffer (50 mM Tris (pH 8.0), 100 mM NaCl, 5% glycerol, 5 mM imidazole, and 1 mM phenylmethylsulfonyl fluoride (PMSF)). After ultracentrifugation at 4° C. at 18,000 rpm for 40 minutes, a soluble lysate was incubated with Ni-NTA resin (Thermo Fisher Scientific) at 4° C. for 2 hours, and purified using a poly-prep chromatography column (Bio-Rad). The column-bound proteins were eluted using a lysis buffer (containing 50 mM Tris (pH 8.0), 50 mM NaCl, 5% glycerol, 300 mM imidazole, and 1 mM PMSF), and impurities were removed using an ultrafiltration spin column (Millipore). The purity of each fusion protein was measured by SDS-PAGE gel (FIGS. 6A and 6B).

### Example 3: Verification of Internalization of Gene Editing Complex including Fusion Protein into Natural Killer Cells

In order to confirm whether the gene editing complex including the fusion protein prepared in Example 2 is able to be internalized into NK cells without a carrier, the following experiment was performed.

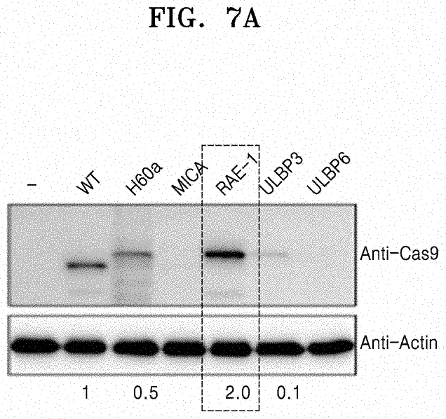

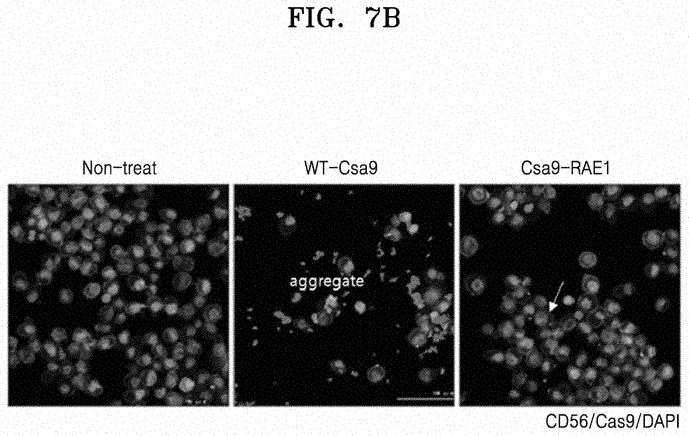

First, the fusion protein was prepared in the form of Cas9 RNP [fusion protein including Cas9 (77.5 pmol)+TracrRNA (100 pmol), 20 min reaction], and then treated to 2.5×10⁵ KHYG1 cells, which is an NK cell line, and delivered to the cells. 24 hours later, to confirm the intracellular delivery efficiency of the gene editing complex, expression levels of Cas9 protein in a cell lysate was examined using a Cas9 antibody (1:1000, CST) by the Western blotting technique described in Example 1. As a result, when the fusion proteins each including H60A, RAE-1, or ULBP3 among the NKG2D ligands were used, internalization of the complex into NK cells was observed, and in particular, RAE-1 exhibited a remarkably excellent internalization into the cells (FIG. 7A). In addition, the cells, into which the Cas9-RAE1 fusion protein complex was introduced, were stained with CD56-APC antibody (1:100, Biolegend) which is an NK cell marker, a DAPI solution (300 nM) which is a nuclear staining solution, and a Cas9 antibody (1:100, CST). As a result of imaging using Lionheart (BioTek) live cell imaging instrument, internalization of Cas9 protein into NK cells was observed (FIG. 7B).

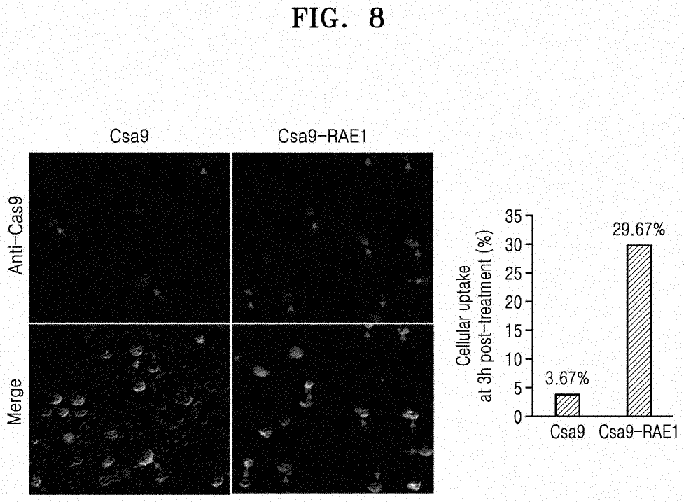

Next, 5×10⁵ NK92 cells were also treated with Cas9 RNP in the same manner, and 6 hours later, the cells were stained with a DAPI solution (300 nM) which is a nuclear staining solution, and a Cas9 antibody (1:100, Abcam). Imaging was performed using Lionheart (BioTek) live cell imaging instrument, and statistical analysis was performed using the instrument software. As a result, it was confirmed that the fusion protein including RAE-1 as the NKG2D ligand and Cas9 showed remarkably excellent efficiency of internalization of the gene editing complex into NK cells (FIG. 8).

The above results taken together, it can be seen that the gene editing complex prepared using the fusion protein including Cas9 and the NKG2D ligand may be effectively internalized into NK cells without a separate carrier.

### Example 4: Examination of Gene Editing Efficiency of Complex including Fusion Protein

To examine whether the gene editing complex including the fusion protein prepared in Example 2 is able to knock-out or knock-in a gene without a carrier, the following experiment was performed.

### 4-1. Examination of Gene Knock-Out

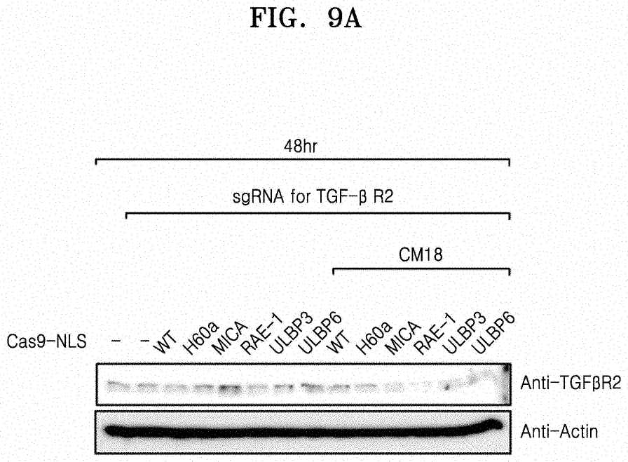

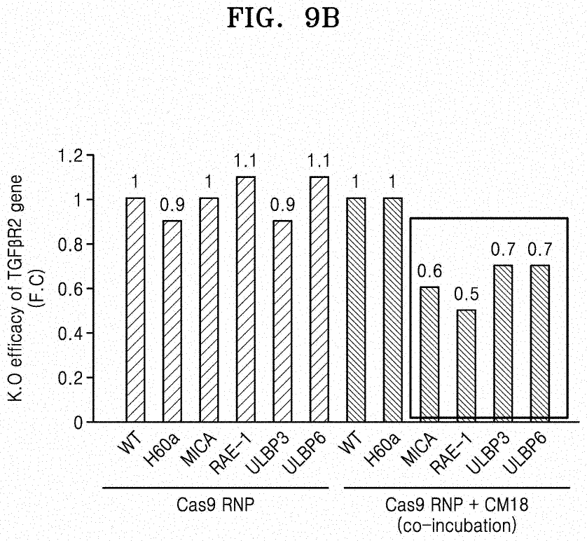

Cas9 RNP was prepared by reacting the fusion protein (38.75 pmol, about 7.5 μg) including Cas9, NLS, and NKG2D ligand with sgRNA (40 pmol) targeting TGFbR2 gene at room temperature for 20 minutes, and then treated to 2.5×10⁵ KHYG1 cells. 48 hours later, TGFbR2 knock-out efficiency was examined by Western blotting. In addition, when preparing Cas9 RNP, CM18 peptide (0.5 nmol, Anygen) which is an endosomal escape peptide was additionally co-cultured for comparison. Band intensity was quantified using a Biorad software. As a result, it was confirmed that the Cas9 RNP prepared by further co-culturing the endosomal escape peptide showed remarkably excellent TGFbR2 knock-out efficiency, as compared with Cas9 RNP prepared using only the fusion protein (including Cas9, NLS, and NKG2D ligand). In particular, when RAE-1 was included, better efficiency was observed (FIGS. 9A and 9B).

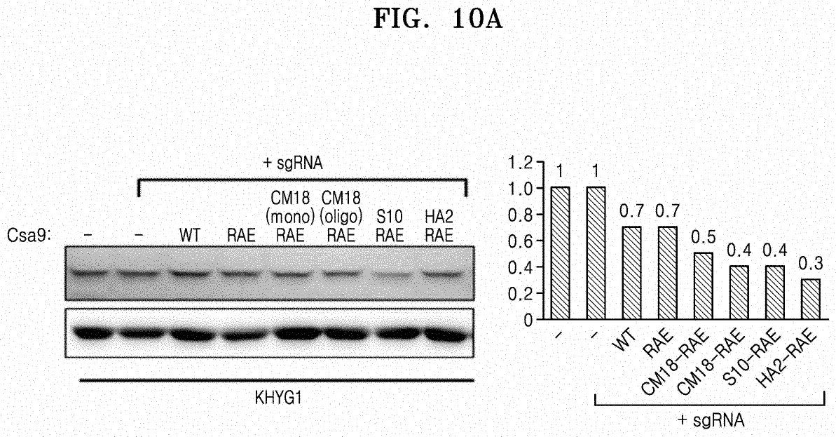

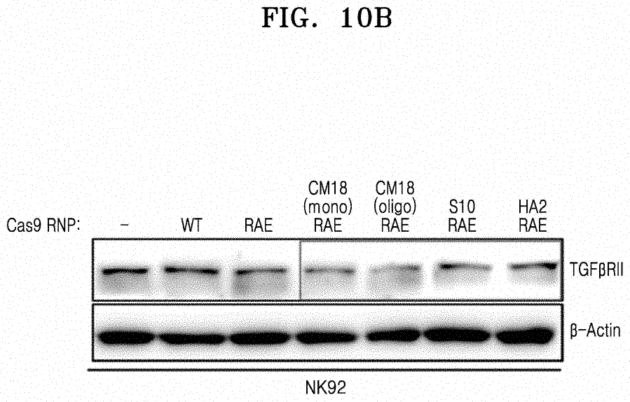

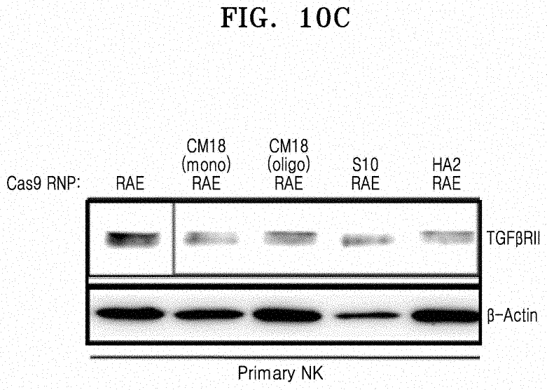

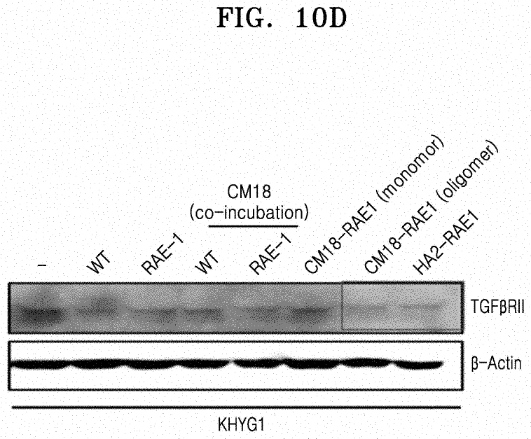

Next, Cas9 RNP prepared using a fusion protein including Cas9, NLS, NKG2D ligand (using RAE-1 as the NKG2D ligand), and endosomal escape peptide was treated to KHYG1, NK92, and primary NK cells, respectively, in the same manner as above. TGFbR2 knock-out efficiency was examined by Western blotting. As a result, remarkably excellent TGFbR2 knock-out efficiency was also observed when the endosomal escape peptide was included in the fusion protein (FIGS. 10A to 10C).

Next, TGFbR2 knock-out efficiency of Cas9 RNP prepared by using RAE-1 as the NKG2D ligand and co-culturing with a separate endosomal escape peptide as described above and Cas9 RNP prepared by using the fusion protein including the endosomal escape peptide was examined by Western blotting. As a result, the complex prepared by inserting the endosomal escape peptide into the fusion protein showed excellent TGFbR2 knock-out efficiency, as compared with the complex prepared by co-culturing with the separate endosomal escape peptide (FIG. 10D).

### 4-2. Examination of Gene Knock-In

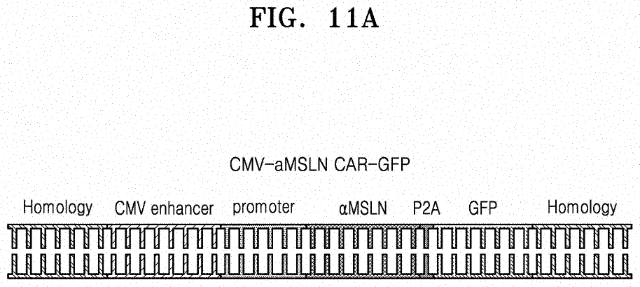

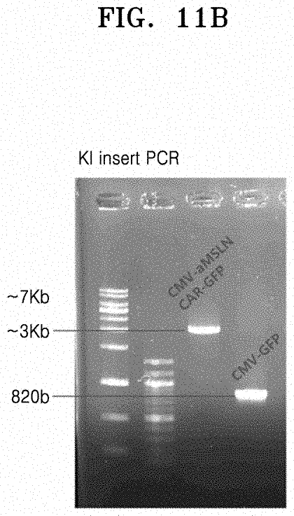

First, to construct a donor DNA fragment for gene knock-in, an expression vector including a CMV promoter, anti-MSLN CAR, P2A, and GFP was prepared, and homology regions at both ends of genomic sequences recognized by TGFbR2 sgRNA were amplified by PCR, respectively, and introduced into the vector to prepare a vector. Next, a KI insert donor DNA fragment was subjected to PCR using the vector as a template and primers capable of amplifying the same, and used after purifying using an elution kit (FIGS. 11A and 11B).

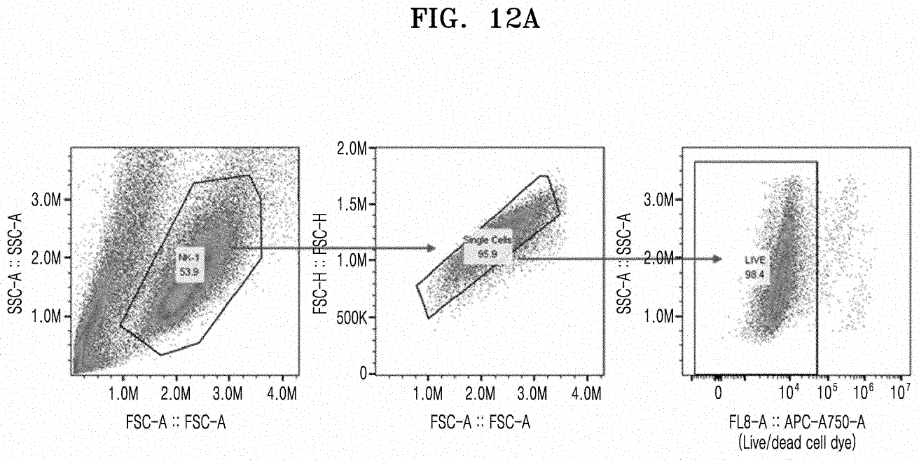

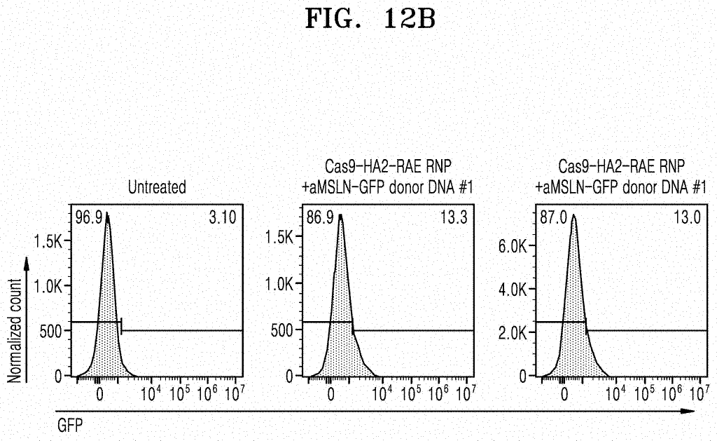

Next, to examine the knock-in efficiency of the gene editing complex of the present disclosure, Cas9-HA2-RAE1 fusion protein (150 pmol), sgRNA (120 pmol) targeting the TGFbR2 gene, and KI insert donor DNA fragment (500 ng) expressing anti-MSLN CAR-GFP were allowed to react at room temperature for 20 minutes to prepare Cas9 RNP +donor DNA, which was then treated to NK92 cells which is an NK cell line. 72 hours later, GFP expression efficiency was examined by FACS analysis. As a result of the above experiment, it was confirmed that 10% or more gene insertion occurred, as compared with a control group (FIGS. 12A and 12B).

The above experimental results taken together, since the Cas9 fusion protein of the present disclosure includes the NKG2D ligand, it may specifically bind to the NKG2D receptor and may be effectively delivered into cells through a cell endocytosis process without a carrier. Thus, it can be seen that the gene editing complex including the fusion protein may be introduced into cells expressing NKG2D on the cell membrane or natural killer cells without a transporter to effectively perform gene editing processes such as gene knock-out, knock-in, etc.

Since the gene editing complex including the fusion protein of the present disclosure includes the NKG2D ligand capable of binding to NKG2D expressed on the membrane of natural killer cells, it may be specifically delivered to NKG2D receptor-expressing cells or natural killer cells. Since the complex may be effectively delivered into the cells through endocytosis of NKG2D without a carrier, a target gene of natural killer cells, or a target gene or target DNA of NKG2D receptor-expressing cells may be manipulated using the complex.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the figures, it will be understood by those of ordinary skill in the art that various changes in form and details may be made therein without departing from the spirit and scope of the disclosure as defined by the following claims.

What is claimed is:

1. A fusion protein comprising a CRISPR-associated protein (Cas protein) and an NKG2D ligand-derived protein.

2. The fusion protein of claim 1, wherein the NKG2D ligand is one or more selected from the group consisting of UL16 binding protein 3 (ULBP3), MHC Class I Polypeptide-Related Sequence A (MICA), ULBP6, Retinoic acid early-inducible protein 1-beta (RAE1), histocompatibility antigen 60a (H60A), MICB, ULBP1, ULBP2, ULBP4, ULBP5, RAE1α, RAE1γ, RAE1δ, RAE1ϵ, and H60B.

3. The fusion protein of claim 1, wherein the Cas protein is a Cas9 protein, a Cpf1 protein, or a Cas variant protein.

4. The fusion protein of claim 1, further comprising a nuclear localization sequence (NLS).

5. The fusion protein of claim 1, further comprising an endosomal escape peptide.

6. The fusion protein of claim 5, wherein the endosomal escape peptide is an HA2 peptide, an S10 peptide, or a CM18 peptide.

7. A polynucleotide encoding the fusion protein of claim 1.

8. An expression vector comprising the polynucleotide of claim 7.

9. A gene editing complex comprising a fusion protein comprising a CRISPR-associated protein (Cas protein) and an NKG2D ligand-derived protein; and guide RNA.

10. The gene editing complex of claim 9, further comprising an endosomal escape peptide.

11. The gene editing complex of claim 10, wherein the endosomal escape peptide is comprised in the fusion protein, or comprised as a separate protein in the complex.

12. The gene editing complex of claim 9, wherein the guide RNA is a dual RNA comprising CRISPR RNA (crRNA) and transactivating crRNA (tracrRNA), or a single-chain guide RNA (sgRNA) comprising a portion of the crRNA and tracrRNA and hybridizing with the target DNA.

13. The gene editing complex of claim 9, wherein the complex is self-assembled by the fusion protein to form a complex with the guide RNA.

14. A composition for editing a target DNA or a target gene specifically to NKG2D receptor-expressing cells or natural killer cells, the composition comprising the gene editing complex of claim 9.

15. The composition of claim 14, wherein the composition is carrier-free.

16. A method for treating cancer, comprising administering to a subject a pharmaceutical composition comprising the complex of claim 9.

17. The method of claim 16, wherein the cancer is liver cancer, lung cancer, pancreatic cancer, non-small cell lung cancer, colon cancer, bone cancer, skin cancer, head or neck cancer, skin or intraocular melanoma, uterine cancer, ovarian cancer, rectal cancer, gastric cancer, perianal cancer, colon cancer, breast cancer, fallopian tube carcinoma, uterine endometrial carcinoma, cervical carcinoma, vaginal carcinoma, vulva carcinoma, Hodgkin's disease, esophageal cancer, small intestine cancer, endocrine cancer, thyroid cancer, parathyroid cancer, adrenal gland cancer, soft tissue sarcoma, urethral cancer, penile cancer, prostate cancer, chronic or acute leukemia, lymphocytic lymphoma, bladder cancer, kidney or ureter cancer, renal cell carcinoma, renal pelvic carcinoma, central nervous system (CNS) tumor, primary CNS lymphoma, spinal tumor, brainstem glioma, or pituitary adenoma.
